(19) United States
(12) Patent Application Publication
(10) Pub. No.: US 2024/0307556 A1
(43) Pub. Date: Sep. 19, 2024

(54) COMPOSITIONS AND METHODS FOR REVERSE GENE THERAPY

(71) Applicant: Maritime Therapeutics, Inc., Carlisle, MA (US)

(72) Inventor: Michael Heartlein, Carlisle, MA (US)

(21) Appl. No.: 18/002,567

(22) PCT Filed: Jul. 2, 2021
(86) PCT No.: PCT/US2021/040316

§ 371 Date: Dec. 20, 2022

Related U.S. Application Data
(60) Provisional application No. 63/047,575, filed on Jul. 2, 2020.

(51) Int. Cl. A61K 48/00; C12N 15/90

(52) U.S. Cl. CPC A61K 48/005; C12N 15/907; C12N 2800/22

(57) ABSTRACT

The present invention provides, among other things, mRNA constructs and compositions and methods for reverse gene therapy, including administering to a subject in need of treatment a mRNA gene transfer construct comprising a reverse complement sequence encoding a protein of interest and a sequence encoding a human L1 retro-element.

### CROSS REFERENCE TO RELATED APPLICATION

This application claims priority to U.S. Provisional Application Ser. No. 63/047,575, filed Jul. 2, 2020, which is incorporated by reference herein in its entirety.

### BACKGROUND

Conventional gene therapy methods are based on administration of nucleic acid molecules to a patient. However, conventional gene therapy methods carry the risk of unwanted insertion into the patient's genome and require potentially immunogenic components to facilitate transgene delivery. Additionally, current methods for gene delivery limit gene size due to packaging constraints. Thus, there is a need for the delivery of biotherapeutic agents that address the difficulties surrounding intracellular processing of nucleic acids encoding polypeptides, and thus the optimization of protein expression from the delivered therapeutic agents.

### SUMMARY OF THE INVENTION

The present invention addresses this need by providing compositions and methods for gene delivery using intrinsically non-immunogenic components capable of active transport of a reverse gene transfer construct to the nucleus. The present invention is based, in part, on the insight that efficient gene delivery may be achieved using a gene transfer construct comprising a reverse complement sequence for a protein of interest coupled with L1 retro elements that leverage endogenous human gene processing machinery. Including the protein of interest in the reverse orientation ensures that the gene is not prematurely activated. In some embodiments, provided herein are compositions and methods for single dose, non-viral, permanent gene therapy that uses “XE” sequence elements to drive high-level, stable, gene expression from a chromosomally targeted transgene. The present invention combines the versatility, efficiency and potency of mRNA transcript therapies with the durability of gene therapy and gene editing technologies.

In one aspect, the present invention provides a composition comprising an mRNA gene transfer construct comprising a reverse complement sequence encoding a protein of interest; and a sequence encoding a human L1 retro-element. In another aspect, the present invention provides a composition comprising an mRNA gene transfer construct comprising a sequence encoding a protein of interest; and a sequence encoding a human L1 retro-element. As used herein, a sequence encoding a protein of interest can be present on the gene transfer construct in the forward orientation.

In some embodiments, the gene transfer construct comprises a matrix attachment region (MAR) motif.

In some embodiments, the MAR motif comprises a sequence selected from TT(A/T)T(T/A)TT(T/A)TT or AATAAA(T/C)AAA. In some embodiments, the MAR motif comprises a human X Chromosome fragment surrounding hypoxanthine-guanine phosphoribosyltransferase (HPRT) exons II and III.

In some embodiments, the gene transfer construct comprises a reverse complement sequence of the MAR motif.

In some embodiments, the gene transfer construct further comprises a topoisomerase II consensus sequence.

In some embodiments, the topoisomerase II consensus sequence comprises GTN(A/T)A(T/C)ATINATNN(G/A).

In some embodiments, the gene transfer construct comprises a reverse complement sequence of the topoisomerase II consensus sequence.

In some embodiments, gene transfer construct comprises a eukaryotic origin of replication.

In some embodiments, the gene transfer construct comprises a promoter, an enhancer and/or an intron.

In some embodiments, the promoter, enhancer and/or intron are derived from Cytomegalovirus (CMV).

In some embodiments, the gene transfer construct comprises a reverse complement sequence of the promoter, enhancer and/or intron.

In some embodiments, the sequence encoding a human L1 retro-element is a mRNA transcript. In some embodiments, the human L1 retro-element comprises a nuclear localization signal (NLS).

In some embodiments, the human L1 retro-element comprises a RNA binding domain.

In some embodiments, the human L1 retro-element comprises an ORF2 reverse transcriptase domain. In some embodiments, the human L1 retro-element comprises an ORF2 endonuclease domain. In some embodiments, the human L1 retro-element comprises ORF2.

In some embodiments, the human L1 retro-element comprises an ORF2 reverse transcriptase and does not comprise an ORF2 endonuclease domain.

In some embodiments, the human L1 retro-element comprises ORF1.

In some embodiments, the mRNA gene transfer construct and the sequence encoding a human L1 retro-element are present on a single transcript.

In some embodiments, the mRNA gene transfer construct and the sequence encoding a human L1 retro-element are present on different transcripts.

In some embodiments, the gene encoding the protein of interest is greater than about 4.5 Kb. In some embodiments, the gene encoding the protein of interest is between about 4.5-15 Kb.

In some embodiments, the composition comprises a DNA primer for self-priming.

In some embodiments, the composition comprises an inverted terminal repeat sequence for self-priming.

In some embodiments, the mRNA is codon optimized.

In some embodiments, the mRNA comprise a native CAP 5′ structure.

In some embodiments, the mRNA comprise an enzymatically derived poly A 3′ end.

In some embodiments, the protein of interest is a therapeutic protein for a single gene deficiency disease, an infectious disease, or cancer.

In some embodiments, the mRNA gene transfer construct and the sequence encoding a human L1 retro-element are encapsulated in a nanoparticle delivery vehicle. In some embodiments, the mRNA gene transfer construct and the sequence encoding a human L1 retro-element are present on a single transcript. In some embodiments, the mRNA gene transfer construct and the sequence encoding a human L1 retro-element are present on different transcripts.

In some embodiments, the nanoparticle delivery vehicle is a lipid nanoparticle (LNP).

In some embodiments, the mRNA gene transfer construct and the sequence encoding a human L1 retro-element are encapsulated in an exosome.

In one aspect, the present invention provides a method for gene delivery comprising administering a composition described herein to a subject in need of treatment.

In one aspect, the present invention provides a method of treating a disease comprising a method of gene delivery comprising administering a mRNA gene transfer construct comprising a reverse complement sequence encoding a protein of interest; and administering a sequence encoding a human L1 retro-element.

In one aspect, the present invention provides a method of treating a disease comprising a method of gene delivery comprising administering a mRNA gene transfer construct comprising a sequence encoding a protein of interest; and administering a sequence encoding a human L1 retro-element.

In some embodiments, the disease is a single gene deficiency disease, an infectious disease, or a cancer.

In one aspect, the present invention provides a method for gene delivery comprising administering a mRNA gene transfer construct comprising a reverse complement sequence encoding a protein of interest; and administering a sequence encoding a human L1 retro-element.

In some embodiments, the method comprises administering the mRNA gene transfer construct and the sequence encoding a human L1 retro-element simultaneously.

In some embodiments, the method comprises administering the mRNA gene transfer construct and the sequence encoding a human L1 retro-element sequentially.

In some embodiments, the gene transfer construct is integrated into the host chromosome.

In some embodiments, the gene transfer construct remains extrachromosomal.

In some embodiments, the mRNA gene transfer construct and/or the sequence encoding a human L1 retro-element are encapsulated in a nanoparticle delivery vehicle.

In some embodiments, the method comprises administering the nanoparticle delivery vehicle intravenously, intrathecally, by aerosolization or by intramuscular injection.

In some embodiments, the method comprises administering the nanoparticle delivery vehicle using ex vivo gene transfer to T cells, induced pluripotent stem cells, stem cells, bone marrow stem cells or other blood cells

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1 depicts an exemplary process for RGT delivery and transgene integration into a host chromosome.

FIG. 2 illustrates an exemplary nucleic acid construct comprising XE sequences derived from HGPRT1.

FIG. 3 illustrates an exemplary RGT single transcript construct.

FIG. 4 illustrates exemplary RGT mRNA transcripts.

FIG. 5 illustrates an exemplary MAR motif present in HGPRT1.

FIG. 6 illustrates exemplary RGV constructs in cis (L1-ORFs on the same script) and in trans (L1 ORFs and gene of interest on different transcripts) configurations and in vitro transfection results.

FIG. 7 illustrates an exemplary PCR results of colonies transfected with RGV constructs in cis (L1-ORFs on the same script) or in trans (L1 ORFs and gene of interest on different transcripts) configurations.

FIG. 8 illustrates an exemplary RGV construct comprising L1-ORFs, XE sequences and gene of interests and an exemplary RGV construct comprising L1 ORF2 and gene of interest without XE sequences and L1 ORF1. The cells transfected with respective RGV constructs are shown.

FIG. 9 shows an exemplary bar graphs illustrating protein expression driven by transfected RGV constructs and the respective PCR analysis.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, %12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

L1 retro-element or L1 retro-unit: As used herein, the terms “L1 retro-element” or “L1 retro-unit” as used herein refer to one or more components of human L1 retrotransposons. The terms “L1 retro-element” and “L1 retro-unit” are used interchangeably. In some embodiments, the L1 retro-element refers to an mRNA sequence selected from ORF1, ORF2 endonuclease domain and/or ORF2 reverse transcriptase domains. The function of the L1 retro-element is to supply protein factors to facilitate the nuclear transfer and processing of the gene transfer construct. In some embodiments, processing of the gene transfer construct involves reverse transcription by the protein encoded by ORF2. In some embodiments, processing of the gene transfer construct involves reverse transcription and chromosomal integration of the gene transfer construct by the protein encoded by ORF2.

Local distribution or delivery: As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient's circulation system.

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). Thus, the term “liposome” as used herein encompasses both lipid and polymer based nanoparticles. In some embodiments, a liposome suitable for the present invention contains cationic or non-cationic lipid(s), cholesterol-based lipid(s) and PEG-modified lipid(s).

messenger RNA (mRNA): As used herein, the term “messenger RNA (mRNA)” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acid. As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or rnalonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, nucleic acid constructs, compositions and methods for reverse gene therapy (RGT). The present invention provides a method for gene delivery, including administering to a subject in need of treatment a gene transfer construct comprising a reverse complement sequence encoding a protein of interest (RGV) and a sequence encoding a human L1 retro-element. Accordingly, the present invention provides for single dose, non-viral, non-immunogenic gene therapy using natural human gene processing and gene transfer factors. Additionally, as compared to the traditional gene therapy, the present invention allows cost-efficient and gram-scale commercial manufacturing due to cell-free biosynthetic drug product and removes limitations of gene size. Notably, immunogenicity is not a barrier as the present invention uses intrinsically non-immunogenic components, allowing for additional dosing if necessary.

In one aspect, the present invention provides an mRNA gene transfer construct comprising a reverse complement sequence encoding a protein of interest (RGV) and a sequence encoding a human L1 retro-element.

In another aspect, the present invention provides a composition comprising an mRNA gene transfer construct comprising a sequence encoding a protein of interest; and a sequence encoding a human L1 retro-element. As used herein, a sequence encoding a protein of interest can be present on the gene transfer construct in the forward orientation.

### Reverse Gene Therapy (RGT)

Reverse gene therapy (RGT) is a nucleic acid therapy that facilitates effective gene transfer to a host. Native and plasmid DNA-based gene transfer mechanisms require RNA or DNA to be delivered to the nucleus for primary transcript generation and processing in the nucleus followed by transport back to the cytoplasm before translation of gene processing proteins can occur. With RGT, gene transfer and processing proteins are translated directly upon delivery to the cytoplasm following release of the transcripts from a delivery vehicle (e.g., a lipid nanoparticle (LNP)).

In some embodiments, RGT comprises DNA constructs. In some embodiments, RGT comprises RNA constructs. In some embodiments, RGT comprises mRNA constructs. In some embodiments, RGT is provided on a single transcript. In some embodiments, RGT is provided as a multi-transcript composition.

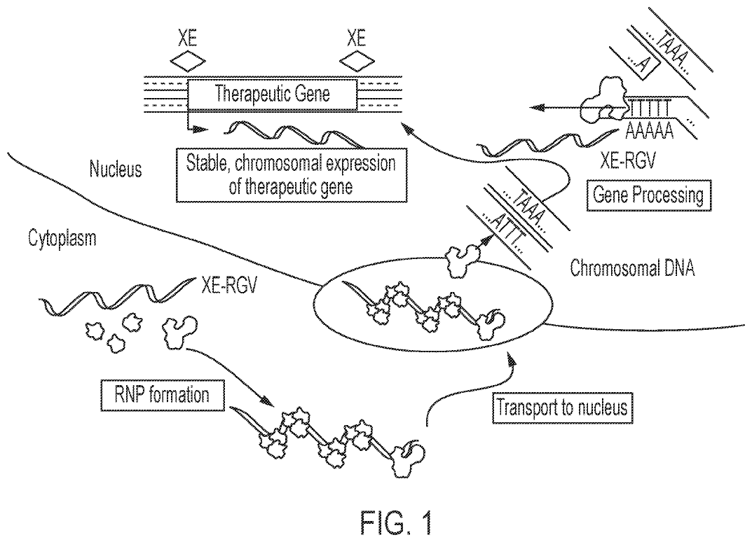

In some embodiments, RGT incorporates the reverse transcriptase (RT) activity of ORF2p. In some embodiments, ORF2 reverse transcriptase is co-packaged and delivered with the mRNA encoding a reverse gene transfer transcript. RT activity converts the RGV-RNA into RGV-DNA. ORF2p then directs the RGV-DNA to be permanently integrated into, and expressed from, the chromosome. As shown in FIG. 1, the mRNA gene transfer construct comprising a reverse complement sequence encoding a protein of interest (XE-RGV) interacts with ORF1 and ORF2 proteins to facilitate nuclear transport, integration and reverse transcription.

For some applications integration into the chromosome may not be desired. Accordingly, in some embodiments, RGT comprises a pre-primed RGV or an RGV capable of self-priming the ORF2p reverse transcriptase. In some embodiments, RGT comprises an endonuclease-deficient variant of ORF2p such that the reverse transcribed RGV will remain extrachromosomal.

### Reverse Gene Therapy (RGT) Constructs

The reverse gene vector (RGV) comprises an mRNA gene transfer construct comprising a reverse complement sequence encoding a protein of interest. The mRNA for the protein of interest includes the gene in reverse and is therefore non-coding until it is reverse transcribed. The RGV also comprises XE sequences to facilitate site-specific chromosomal integration, targeting, and/or homologous recombination integration into the host chromosome.

### X-E Sequences

XE sequences are human chromosomal DNA sequences selected based upon their ability to reproducibly confer high level, stable transgene expression. These elements include sequences that can function by insulating transgenes from chromosomal position effects and factors that attenuate gene expression. The RGT system can be used for site-specific chromosomal integration, targeting and homologous recombination. The “X-E” elements comprise matrix attachment regions (MAR) (also referred to as scaffold associated regions (SAR)). Because these types of elements promote homologous recombination, the present invention is based in part on the use of these properties to develop RGT for highly efficient gene targeting and expression.

### Matrix Attachment Regions (MARs)

The stability and/or expression of the RGV can be increased by insertion of MAR (Matrix Associated Region) or SAR (Scaffold Associated Region) elements in the gene transfer construct. These SAR or MAR regions are AT-rich sequences and enable may function to anchor the reverse gene vector to the matrix of the cell chromosome and regulating the processing of the polynucleotide encoding a reverse complement sequence for the protein of interest. The MAR/SAR (S/MAR) sequences may also function by stimulating expression of the transgene and improving chromatin accessibility.

A S/MAR sequence element can mediate the attachment of specific areas of interphase nuclear chromatin to the lamina of the nuclear matrix. The higher order structure of eukaryotic chromosomes include independent loop domains, which are thought to be separated from each other by the periodic attachment of MARs onto the nuclear matrix. MARs can thereby serve as insulators of a transcription unit in a naturally occurring gene. The general attributes of MARs have been summarized in Boulikas, 1993, J. Cell Biochem. 52:14-22 and are reviewed in Allen et al., 2000, Plant Mol. Biol. 43:361-76). MARs often include potential origins of replication, relatively long A-T-rich stretches having, e.g., topoisomerase II binding sites and/or palindromic sequences. Some classes of MARs contain CT-rich stretches or may be enriched in TG-motifs. In addition, MAR's can include transcription factor binding sites and can contain potentially curved or kinked DNA. A construct described herein can include at least one MAR. Where more than one, e.g., two, MAR's are employed in a construct described herein, e.g., flanking 5′ and 3′ of a nucleic acid encoding a polypeptide, they may be the same or different. Preferred MARs described herein are mammalian MAR's, preferably human MAR's, although non-mammalian MAR's can also be used if they function in a mammalian cell

In some embodiments, the gene transfer construct comprises a matrix attachment region (MAR) or SAR (Scaffold Associated Region) (collectively S/MAR). In some embodiments, S/MARs comprise one or more features selected from the group consisting of OriC, AT richness, kinked and curved DNA, TG richness, MAR signature and Topoisomerase-II sites. In some embodiments, the S/MAR comprises a specific motif characterized as having one or more features selected from the group consisting of OriC, AT richness, kinked and curved DNA, TG richness, MAR signature and Topoisomerase-II sites.

In some embodiments, the gene transfer construct comprises an OriC motif selected from the group consisting of ATTA or ATTTA or ATTTTA. In some embodiments, the gene transfer construct comprises AT rich motif. In some embodiments, the AT rich motif is determined by presence of two WWWWWW (where W is A or T) motifs intervened by 8-12 nt.

In some embodiments, the gene transfer construct (e.g., RGV) comprises a matrix attachment region (MAR) motif. In some embodiments, the gene transfer construct comprises a reverse complement sequence of a MAR motif. In some embodiments, the MAR motif comprises a sequence selected from TT(A/T)T(T/A)TT(T/A)TT or AATAAA(T/C)AAA.

In some embodiments, the MAR comprises an AT rich sequence. In some embodiments, the MAR comprises one or more repeats (e.g., 2 repeats, 3 repeats, 4 repeats, 5 repeats, 6 repeats, 7 repeats, 8 repeats, 9 repeats, 10 repeats) of TATTT, TATTTT, TTTATT or subsets thereof. In some embodiments, the MAR comprises a sequence that is at least 80% adenine and thymine. In some embodiments, the MAR comprises a sequence that is at least 85%, at least 86%, at least 87%, at least 88%, at least 89%, at least 90%, at least 91%, at least 92%, at least 93%, at least 94% or at least 95% adenine and thymine.

In some embodiments, the gene transfer construct comprises a kinked DNA motif selected from the group consisting of TAN3TGN3CA or TAN3CAN3TG or TGN3 TAN3CA or TGN3CAN3TA or CAN3 TAN3TG or CAN3TGN3TA motif (where N is any nucleotide). In some embodiments, the gene transfer construct comprises an curved DNA motif selected from the group consisting of AAAAN7AAAAN7AAAA or TTTTN7TTTTN7TTTT or TTTAAA (where N is any nucleotide). In some embodiments, the gene transfer construct comprises a TG rich motif. In some embodiments, the TG rich motif comprises TGTTTTG or TGTTTTTTG or TTTTGGGG. In some embodiments, the gene transfer construct comprises a MAR signature determined by the presence of a bipartite sequence containing AATAAYAA and AWWRTAANNWWGNNNC (where W is A or T, Y is pyrimidine, R is purine and N is any nucleotide).

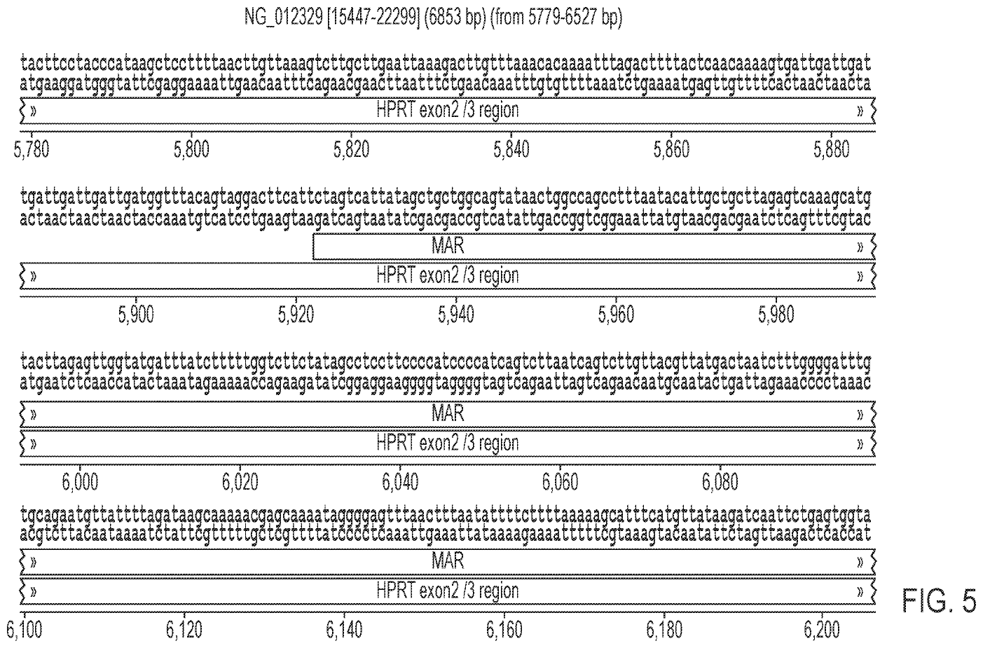

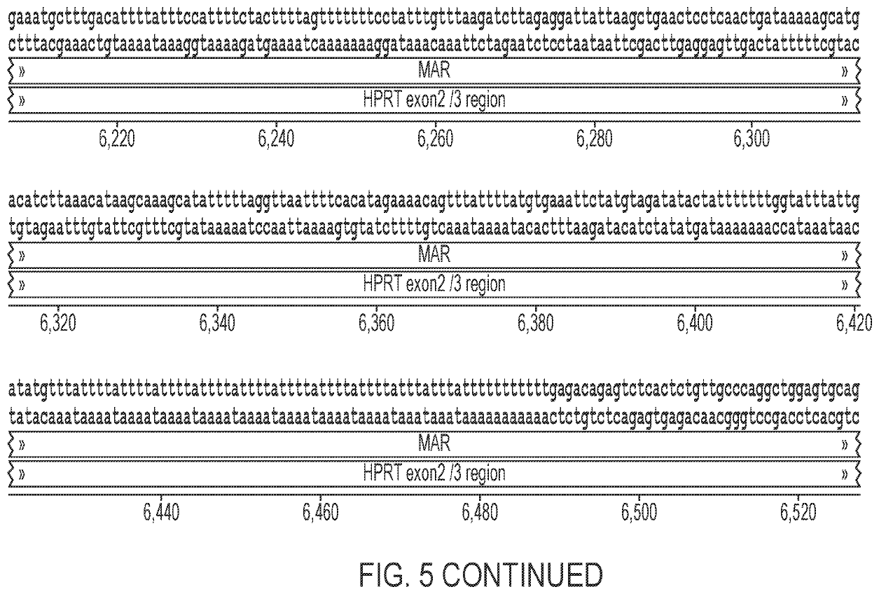

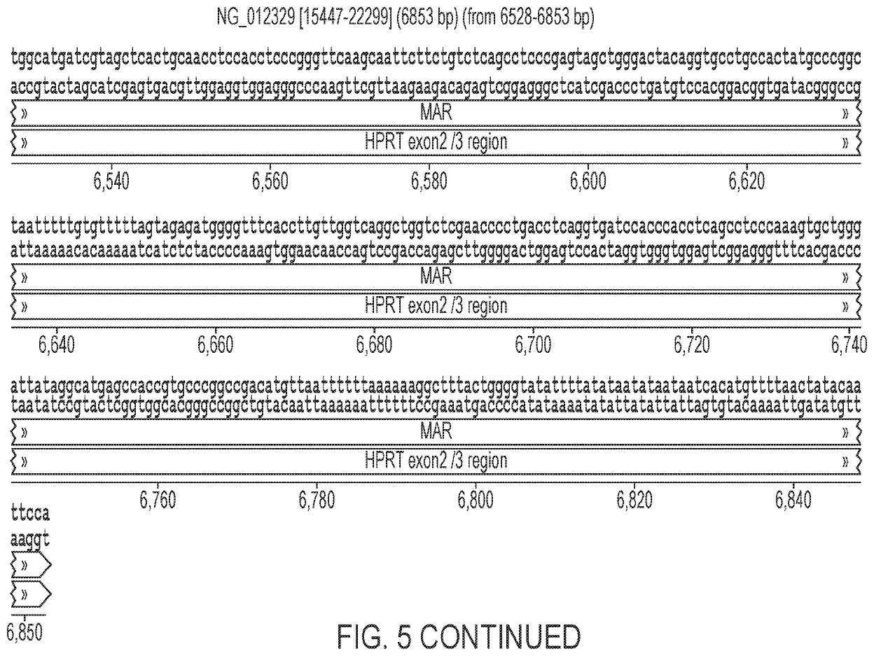

In some embodiments, the MAR motif comprises a human X Chromosome fragment surrounding hypoxanthine-guanine phosphoribosyltransferase (HPRT) exons II and III. In some embodiments, the MAR comprises an HPRT sequence that is a 6.8 Kb fragment of NG_012329 [15447-22299] (6853 bp), as shown in FIG. 5.

In some embodiments, the MAR comprises a sequence derived from a gene selected from Heat Shock Cognate 80 gene of tomato (HSC80), human apolipoprotein B, human interferon beta (IFN-β), or β-globin.

In some embodiments, the MAR is human β-interferon MAR (βI MAR), as described in, e.g., Bode et al. (1988) Biochemistry 27:4706-4711, and US200303224477A1 which are hereby incorporated by reference in their entirety. Other MAR's that can be used in the constructs described herein include the keratin 18 (K18) MAR's (U.S. Pat. No. 5,840,555; Neznanov et al., 1993, Mol. Cell Biol. 13:2214-2223); chicken lysozyme 5′ MAR, which is known to function in mammalian cells (Phi-Ban et al., 1990, Mol. Cell Biol. 10:2302-2307); human β-globin 5′ MAR (Yu et al., 1994, Gene 139:139-145); MAR from T cell receptor beta; and human chromosome 19 MAR's, e.g., GenBank Accession Numbers Z35279, Z35288, Z35291, Z35290, Z35220, Z35221, Z35222, Z35223, and Z35224 (Nikolaev et al., 1996, Nuc. Acids Res. 24:1330-1336).

In some embodiments, the MAR sequence is at least 80%, (e.g., at least 85%, at least 90%, or at least 95%) identical to a human MAR sequence.

### Topo II

Topoisomerase II is involved in controlling the topological structure of DNA and associates with the nuclear matrix. In some embodiments, the gene transfer construct (e.g., RGV) comprises a topoisomerase II consensus sequence. In some embodiments, the gene transfer construct comprises a Topoisomerase II binding site. In some embodiments, the Topoisomerase II binding site comprises RNYNNCNNGYNGKTNYNY or GTNWAYATTNATNNR (where W is A or T, Y is pyrimidine, R is purine and N is any nucleotide). In some embodiments, the gene transfer construct (e.g., RGV) comprises a sequence with homology to the in vitro cleavage consensus of Drosophila topoisomerase II. In some embodiments, the topoisomerase II consensus sequence comprises GTN(A/T)A(T/C)ATINATNN(G/A). In some embodiments, the gene transfer construct comprises a reverse complement sequence of the topoisomerase II consensus sequence.

### Functional Sequence Elements

In some embodiments, gene transfer construct further comprises an origin of replication. In some embodiments, the origin of replication comprises sequences derived from a eukaryotic origin of DNA replication to facilitate replication in a eukaryotic cell. The nature of the eukaryotic replication origin sequences to be used will depend upon the application contemplated for the retrotransposon. For example, it may be necessary in some instances to include an origin of DNA replication which facilitates replication of the DNA molecule in a low copy number. Alternatively, a high copy number of the DNA molecule in cells may be required in which case an origin of DNA replication capable of yielding a high copy number of DNA molecules is preferable. Similarly, it may be necessary to direct replication of the DNA molecule to the nucleus of the cell, and it may be necessary that such replication be episomal in nature. Origins of replication which are useful for the generation of either low copy number or high copy number, include, as examples, oriP driven by the EBNA1 protein or a papillomavirus origin of DNA replication which generate approximately 10-20 copies of DNA per cell (high copy number) and mammalian artificial chromosomes which generate 1-2 copies per cell (low copy number).

In some embodiments, the gene transfer construct includes a eukaryotic origin of DNA replication. In some embodiments, the gene transfer construct comprises the core autonomously replicating sequences (ARS) of yeast. In some embodiments, the eukaryotic origin of DNA replication is selected from a sequence residing in intron 3 of the human HPRT gene. The origin of replication may facilitate replication of the reverse transcribed gene transfer construct. As such it may be desirable to direct replication of the reverse transcribed gene transfer construct to the nucleus of the cell so that such replication is extrachromosomal in nature. In some embodiments, the origin of replication is about 1.8 Kb upstream of the MAR (in the intron between exons 2 and 3) NG_012329 [15447-22299] (6853 bp) as shown in FIG. 5.

In some embodiments, the origin of replication comprises sequences derived from a virus, such as, but not limited to, Epstein Barr virus (EBV) comprising oriP and EBNA1 or a polyoma-based virus comprising the polyomavirus origin of replication and a polyomavirus enhancer sequence. In some embodiments, the origin of replication comprises sequences derived from adeno-associated virus, lentivirus, parvovirus, herpes simplex virus, retroviruses, or poxviruses.

In some embodiments, the gene transfer construct comprises a prokaryotic origin of replication. In some embodiments, the origin of replication may also be added to the construct along with an antibiotic resistance gene. Such sequences facilitate replication of the gene transfer construct in prokaryotic cells, thereby facilitating the generation of large quantities of DNA for insertion to the desired eukaryotic cell genome. A prokaryotic origin of DNA replication may also be added along with an antibiotic resistance gene to facilitate growth of the construct in prokaryotic cells. Examples of prokaryotic origins of DNA replication suitable for use include, but are not limited to, the ColEI and pA15 origins of DNA replication. These origins of replication (ori's) are on the not within the sequence to be inserted.

In some embodiments, the RGV comprises a promoter, enhancer and/or intron are derived from Cytomegalovirus (CMV). In some embodiments, the RGV comprises a reverse complement sequence of the promoter, enhancer and/or intron.

ORFP comprises both nuclear localization sequences (NLS) and RNA binding domains. NLS facilitate nuclear uptake actively through interaction with nuclear transport machinery (e.g., nuclear pore proteins KPNA2 and KPNB1). In some embodiments, the human L1 retro-element comprises a nuclear localization signal (NLS).

The RGT transcripts described herein comprise mRNA untranslated regions (UTRs). UTRs of a gene are transcribed but not translated. The 5′UTR starts at the transcription start site and continues to the start codon but does not include the start codon; whereas, the 3′UTR starts immediately following the stop codon and continues until the transcriptional termination signal. Without wishing to be bound by theory, the regulatory roles played by the UTRs may improve stability of the nucleic acid molecule and translation. In some embodiments, the regulatory features of a UTR are incorporated into the mRNA of the present invention to enhance the stability of the molecule. The specific features can also be incorporated to ensure controlled down-regulation of the transcript in case they are misdirected to undesired organs sites.

Natural 5′UTRs include features for translation initiation. They harbor signatures such as Kozak sequences which are commonly known to be involved in the process by which the ribosome initiates translation of many genes. Kozak sequences have the consensus CCRCCAUGG, where R is a purine (adenine or guanine) three bases upstream of the start codon (AUG), which is followed by another ‘G’. 5′UTRs may also form secondary structures which are involved in elongation factor binding.

By engineering the features typically found in abundantly expressed genes of specific target organs, one can enhance the stability and protein production of the mRNA of the invention. For example, introduction of 5′ UTR of liver-expressed mRNA, such as albumin, serum amyloid A, Apolipoprotem A-′B/E, transferrin, alpha fetoprotein, erythropoietin, or Factor VIII, could be used to enhance expression of a nucleic acid molecule, such as a mRNA, in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid ceils (C/EBP, AML1, G-CSF, GM-CSF, CD 1 i b, MSR, Fr-1, i-NOS), for leukocytes (CD45, CD18), for adipose tissue (CD36, GLUT4, ACRP30, adiponectin) and for lung epithelial cells (SP-A/B/C/D). In some embodiments, the 5′ UTR comprises a sequence derived from the human γ-actin promoter.

Other non-UTR sequences may be incorporated into the 5′ (or 3′ UTR) UTRs. For example, introns or portions of introns sequences may be incorporated into the flanking regions of the mRNA of the invention. Incorporation of intronic sequences may increase protein production as well as mRNA levels.

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif c-Jun and Myogenin are two well-studied examples of this class. Most proteins binding to the ARBs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNA of the invention. When engineering specific mRNA, one or more copies of an ARE can be introduced to make mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant ceil lines, using mRNA of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

### Gene of Interest

The reverse gene vector (RGV) is a mRNA gene transfer construct comprising a reverse complement sequence encoding a gene for a protein of interest. In some embodiments, RGT mRNAs encode three proteins. Two of these proteins mediate RNA processing and gene transfer and are expressed normally in human tissues (e.g., ORF1, ORF2, and fragments thereof). The third protein expressed from RGT is a therapeutic protein of interest. In some embodiments, the mRNA encodes a protein, such as a therapeutic protein, a deficient protein, or a functional variant of a nonfunctional protein.

In some embodiments, the gene of interest is delivered in vivo. In some embodiments the gene of interest is integrated into the host chromosome for permanent therapeutic gene expression. In some embodiments, the disease therapeutic protein of interest is delivered to and expressed in liver tissues. In some embodiments, RGT is administered intravenously. In some embodiments, RGT is used for in vivo immunotherapy for infectious diseases and cancer. In some embodiments, the RGT is used for gene transfer to the lymphatics via intramuscular injection. In some embodiments, RGT is used ex vivo for gene transfer to T cells for immunotherapies. In some embodiments, RGT is delivered ex vivo for gene transfer to bone marrow stem cells and other blood cells. In some embodiments, the disease is a single gene deficiency disease.

In some embodiments, the protein of interest is a protein useful for replacing a protein that is deficient or abnormal, augmenting an existing pathway, providing a novel function or activity; or interfering with a molecule or organism. In some embodiments, the protein of interest includes, without limitation, antibody-based drugs, Fc fusion proteins, anticoagulants, blood factors, bone morphogenetic proteins, engineered protein scaffolds, enzymes, growth factors, hormones, interferons, interleukins, and thrombolytics. In some embodiments, the protein of interest acts by binding non-covalently to target (e.g., mAbs); affecting covalent bonds (e.g., enzymes); or exerting activity without specific interactions (e.g., serum albumin). In some embodiments, the protein of interest is includes, without limitation, transcription factors, proteins involved in signal transduction, transcriptional activators, transcriptional repressors, G-proteins, kinases, tumor suppressors, or intrabodies. In some embodiments, the protein of interest is a recombinant protein.

In some embodiments, the protein of interest encoded by the mRNA is used to treat conditions or diseases in many therapeutic areas such as, but not limited to, blood, cardiovascular, CNS, poisoning (including antivenoms), dermatology, endocrinology, genetic, genitourinary, gastrointestinal, musculoskeletal, oncology, and immunology, respiratory, sensory and anti-infective. In some embodiments, the therapeutic protein includes, without limitation, vascular endothelial growth factor (VEGF-A, VEGF-B, VEGF-C, VEGF-D), placenta growth factor (PGF), OX40 ligand (OX40L; CD134L), interleukin 12 (IL12). interleukin 23 (IL23), interleukin 36 γ (IL36y), and CoA mutase.

In some embodiments, the protein of interest encoded by the mRNA replaces a protein that is deficient or abnormal. In some embodiments, the therapeutic protein includes, without limitation, alpha 1 antitrypsin, frataxin, insulin, growth hormone (somatotropin), growth factors, hormones, dystrophin, insulin-tike growth factor 1 (IGF1), factor VIII, factor IX, antithrombm III, protein C, β-Gluco-cerebrosidase, Alglucosidase-a, a-l-iduronidase, Iduronate-2-sulphatase, Galsulphase, human a-galactosidase A, α-1-Proteinase inhibitor, lactase, pancreatic enzymes (including lipase, amylase, and protease), adenosine deaminase, and albumin, including recombinant forms thereof.

In some embodiments, the protein of interest augments an existing pathway. In some embodiments, the protein of interest includes, without limitation, Erythropoietin, Epoetin-a, Darbepoetin, granulocyte colony stimulating factor (G-CSF), granulocyte-macrophage colony stimulating factor (GM-CSF), interleukin 11 (IL11), Human follicle-stimulating hormone (FSH), Huma chorionic gonadotropin (HCG), Lutropin-o, Type I alpha-interferon, Interferon-a2a, Interferon-o2b, Interferon-an3, Interferon-β1a, Interferon-β1b, Interferon-/1b, interleukin 2 (IL2), epidermal thymocyte activating factor (ETAF), tissue plasminogen activator (tPA), Urokinase, factor Vila, activated protein C, Salmon calcitonin, human parathyroid hormone peptide (e.g., residues 1-34), incretin mimetic (e.g., exenatide), somatostatin analogue (e.g., octreotide), recombinant human bone morphogenic protein 2 (rhBMP2), Recombinant human bone morphogenic protein 7 (rhBMP7), gonadotropin releasing hormone (GnRH), keratinocyte growth factor (KGF), platelet-derived growth factor (PDGF), Trypsin, and Recombinant B-type natriuretic peptide.

In some embodiments, the protein of interest provides a novel function or activity. In some embodiments, the therapeutic protein includes, without limitation, Botulinum toxin type A, Botulinum toxin type B, collagenase, Human deoxy-ribonuclease I, domase-a, Hyaluronidase, papain, L-Asparaginase, Rasburicase, Lepirudin, Brvalirudin, Streptokinase, and anisoylated plasminogen streptokinase activator complex (APSAC).

In some embodiments, the protein of interest interferes with a molecule or organism. In some embodiments, the therapeutic protein includes, without limitation, anti-VEGFA antibody, anti-EGFR antibody, anti-CD52 antibody, anti-CD20 antibody, anti-HER2/Neu antibody, fusion protein between extracellular domain of human CTLA4 and the modified Fc portion of human immunoglobulin G1, interleukin 1 (IL1) receptor antagonist, anti-TNFa antibody, CD2-binding protein, anti-CD 11a antibody, anti-a4-subunit of a4β1 and 4β7 integrins antibody, anti-complement protein C5 antibody, Antithymocyte globulin, Chimeric (human/mouse) IgG1, Humanized IgG1 mAb that binds the alpha chain of CD25, anti˜CD3 antibody, anti-IgE antibody, Humanized IgG1 mAb that binds the A antigenic site of the F protein of respiratory syncytial virus, HIV envelope protein gpl20/gp41-binding peptide. Fab fragment of chimeric (human/mouse) mAb 7E3 that binds to the glycoprotein TTb/IIIa integrin receptor, and Fab fragments of IgG that bind and neutralize venom toxins.

In some embodiments, the protein of interest or the L1 retro-element according to any of the embodiments described herein encodes a fusion protein.

In some embodiments, the protein of interest is a chimeric antigen receptor (CAR) or an engineered T cell receptor (TCR). In some embodiments, the CAR specifically binds to a target antigen. In some embodiments, the target antigen is a tumor antigen. In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, UPV-specific antigen, human telom erase reverse transcriptase, IGFI receptor, IGF-II, IL-11Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostase specific antigen (PSA), prostate-carcinoma tumor antigen-1 (PCTA-1), prostate-specific antigen, prostein, PSMA, RAGE-1, ROR1, RU1, RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

### L1 Retro-Element

Retrotransposons are naturally occurring DNA elements found in cells from almost all species of animals, plants and bacteria. Retrotransposons are capable of being expressed in cells, can be reverse transcribed into an extrachromosomal element and can reintegrate into another site on the same genome from which they originated. Retrotransposons may be grouped into two classes, the retrovirus-like LTR retrotransposons, and the poly A elements such as human L1 elements, Neurospora TAD elements (Kinsey, 1990, Genetics 126:317-326), I factors from Drosophila (Bucheton et al., 1984, Cell 38:153-163), and R2Bm from Bombyx mori (Luan et al., 1993, Cell 72: 595-605). Poly A elements (also called non-LTR elements) lack LTRs and end with poly A or A-rich sequences (Luan and Eickbush, 1995, Mol. Cell. Biol. 15:3882-3891; Luan et al., 1993, Cell 72:595-605). Poly A retrotransposons can be subdivided into sequence-specific and non-sequence-specific types. L1 is of the latter type being found to be inserted in a scattered manner in all human, mouse and other mammalian chromosomes.

The L1 element (also known as a LINE). A 6.1 kb full-length L1 consensus sequence has the following conserved organization: A 5′ untranslated leader region (UTR) with an internal promoter; two non-overlapping reading frames (ORF1 and ORF2); a 200 bp 3′ UTR and a 3′ poly A tail. ORF1 encodes a 40 kd protein and may serve a packaging function for the RNA (Martin, 1991, Mol. Cell Biol. 11:4804-4807; Hohjoh et al., 1996, EMBO J. 15:630-639), while ORF2 encodes a reverse transcriptase (Mathias et al., 1991, Science 254:1808-1810). ORF1 and ORF2 proteins associate with L1 RNA, forming a ribonucleoprotein particle. Reverse transcription by ORF2 protein results in L1 cDNAs, which are integrated into the genome (Martin, 1991, Curr. Opin. Genet. Dev. 1:505-508).

Some human L1 elements can retrotranspose (express, cleave their target site, and reverse transcribe their own RNA using the cleaved target site as a primer) into new sites in the human genome, leading to genetic disorders. Germ line L1 insertions into the factor VIII and dystrophin gene give rise to hemophilia A and muscular dystrophy, respectively (Kazazian et al., 1988, Nature 332:164-166; Narita et al., 1993, J. Clinical Invest. 91:1862-1867; Holmes et al., 1994, Nature Genetics 7:143-148), while somatic cell L1 insertions into the c-myc and APC tumor suppressor gene are implicated in rare cases of breast and colon cancer, respectively (Morse et al., Nature 333:87-90; Miki et al., 1992, Cancer Research 52:643-645). Thus, L1 is a potential mutagen and L1 retrotransposition is mutagenic. Exemplary engineered L1 retro-elements are described for example in U.S. Pat. No. 6,150,160, which is hereby incorporated by reference in its entirety.

In some embodiments, a human L1 element comprises a 5′ UTR with an internal promoter, one or more non-overlapping reading frames (e.g., ORF1, ORF2, or fragments thereof), a 3′ UTR and a 3′ poly A tail. In some embodiments, the L1 retro-element of the present invention also comprises an endonuclease domain at the L1 ORF2 N-terminus.

In some embodiments, the L1 retro-element is present on the same transcript as the mRNA gene transfer construct. In some embodiments, the mRNA gene transfer construct and the sequence encoding a human L1 retro-element are present on a single transcript. In some embodiments, the mRNA gene transfer construct and the sequence encoding a human L1 retro-element are present on different transcripts.

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

In some embodiments, an mRNA gene transfer construct comprising a reverse complement sequence encoding a protein of interest and a sequence encoding a human L1 retro-element are present in molar ratio of 1:1. In some embodiments, an mRNA gene transfer construct comprising a reverse complement sequence encoding a protein of interest and a sequence encoding a human L1 retro-element are present in molar ratio of between 1:10 and 10:1. In some embodiments, an mRNA gene transfer construct comprising a reverse complement sequence encoding a protein of interest and a sequence encoding a human L1 retro-element are present in molar ratio of 2:1. In some embodiments, an mRNA gene transfer construct comprising a reverse complement sequence encoding a protein of interest and a sequence encoding a human L1 retro-element are present in molar ratio of 1:2.

### Messenger RNA Based RGT Constructs

Conventional gene therapy involves the use of DNA for insertion of desired genetic information into host cells. The DNA introduced into the cell is usually integrated into the genome of one or more transfected cells, allowing for long-lasting action of the introduced genetic material in the host. While there may be perceived benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response.

In contrast to DNA, the use of RNA as a gene therapy agent is substantially safer because RNA is not integrated into the genome of the transfected cell, thus eliminating the concern that the introduced genetic material will disrupt the normal functioning of an essential gene, or cause a mutation that results in deleterious or oncogenic effects. RNA therapy also does not require extraneous promoter for effective translation of the encoded protein, again avoiding possible deleterious side effects. In addition, any deleterious effects that do result from mRNA based on gene therapy would be of limited duration due to the relatively short half-life.

As described herein, RGT comprises a gene of interest in the reverse complement orientation that requires reverse transcription into a DNA molecule, significantly reducing the risk of unwanted integration or premature expression. In some embodiments, mRNA constructs comprise a 5′ cap structure, a 5′ UTR, an ORF1 protein, an ORF2 protein, a 3′ UTR polyA sequence and XE sequences flanking a gene of interest.

In some embodiments, RGT mRNA is packaged in a delivery vehicle that does not have size constraints. In some embodiments, the transfer vehicles of the present invention are capable of delivering large mRNA sequences. In some embodiments, the RGT mRNA is less than 15 kilobases (kb), e.g., less than 14 kb, less than 13 kb, less than 12 kb, less than 11 kb, less than 10 kb, less than 9 kb, less than 8 kb, less than 7 kb, less than 6 kb, or less than 5 kb. In some embodiments, the mRNA transcript ranges from 4.5 kb to 15 kb or more, e.g., mRNA of a size greater than or equal to 4.5 kb, 5 kb, 5.5 kb, 6 kb, 6.5 kb, 7 kb, 7.5 kb, 8 kb, 8.5 kb, 9 kb, 9.5 kb, 10 kb, 10.5 kb, 11 kb, 11.5 kb, 12 kb, 12.5 kb, 13 kb, 13.5 kb, 14 kb, 14.5 kb, or 15 kb.

In some embodiments, the RGT mRNA is between about 4-15 kb, e.g., about 4.5-15 kb, about 4. less than 14 kb, less than 13 kb, less than 12 kb, less than 11 kb, less than 10 kb, less than 9 kb, less than 8 kb, less than 7 kb, less than 6 kb, or less than 5 kb. packaging constraints that AAV gene therapy technologies suffer from (max size ˜4.5 Kb). Most therapeutic genes for RGT therapies are <15 kb. RGT manufacturing uses cell-free, biosynthetic transcription technologies which are not limited by the size of the DNA template.

In some embodiments, mRNA according to the present invention may be synthesized as unmodified or modified mRNA. Typically, mRNAs are modified to enhance stability. Modifications of mRNA can include, for example, modifications of the nucleotides of the RNA. An modified mRNA according to the invention can thus include, for example, backbone modifications, sugar modifications or base modifications. In some embodiments, mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, beta-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, mRNAs (e.g., mRNAs encoding a gene of interest and/or encoding a human L1 retro-element) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, mRNAs (e.g., mRNAs encoding a gene of interest and/or encoding a human L1 retro-element) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, mRNAs (e.g., mRNAs encoding a gene of interest and/or encoding a human L1 retro-element) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs (e.g., mRNAs encoding a gene of interest and/or encoding a human L1 retro-element) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

In some embodiments, mRNAs (e.g., mRNAs encoding a gene of interest and/or encoding a human L1 retro-element) include a 3′ poly(A) tail structure. A poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

### Cap Structure

In some embodiments, mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G.

Naturally occurring cap structures comprise a 7-methyl guanosine that is linked via a triphosphate bridge to the 5′-end of the first transcribed nucleotide, resulting in a dinucleotide cap of m⁷G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

A common cap for mRNA produced by in vitro transcription is m⁷G(5′)ppp(5′)G, which has been used as the dinucleotide cap in transcription with T7 or SP6 RNA polymerase in vitro to obtain RNAs having a cap structure in their 5′-termini. The prevailing method for the in vitro synthesis of capped mRNA employs a pre-formed dinucleotide of the form m⁷G(5′)ppp(5′)G (“m⁷GpppG”) as an initiator of transcription.

To date, a usual form of a synthetic dinucleotide cap used in in vitro translation experiments is the Anti-Reverse Cap Analog (“ARCA”) or modified ARCA, which is generally a modified cap analog in which the 2′ or 3′ OH group is replaced with —OCH₃.

Additional cap analogs include, but are not limited to, a chemical structures selected from the group consisting of m⁷GpppG, m⁷GpppA, m⁷GpppC; unmethylated cap analogs (e.g., GpppG); dimethylated cap analog (e.g., m^{2,7}GpppG), trimethylated cap analog (e.g., m^{2,2,7}GpppG), dimethylated symmetrical cap analogs (e.g., m⁷Gpppm⁷G), or anti reverse cap analogs (e.g., ARCA; m^{7,2′Ome}GpppG, m^{72′d}GpppG, m^{7,3′Ome}GpppG, m^{7,3′d}GpppG and their tetraphosphate derivatives) (see, e.g., Jemielity, J. et al., “Novel ‘anti-reverse’ cap analogs with superior translational properties”, RNA, 9: 1108-1122 (2003)).

In some embodiments, a suitable cap is a 7-methyl guanylate (“m⁷G”) linked via a triphosphate bridge to the 5′-end of the first transcribed nucleotide, resulting in m⁷G(5′)ppp(5′)N, where N is any nucleoside. A preferred embodiment of a m⁷G cap utilized in embodiments of the invention is m⁷G(5′)ppp(5′)G.

In some embodiments, the cap is a Cap0 structure. Cap0 structures lack a 2′-O-methyl residue of the ribose attached to bases 1 and 2. In some embodiments, the cap is a Cap1 structure. Cap1 structures have a 2′-O-methyl residue at base 2. In some embodiments, the cap is a Cap2 structure. Cap2 structures have a 2′-O-methyl residue attached to both bases 2 and 3.

A variety of m⁷G cap analogs are known in the art, many of which are commercially available. These include the m⁷GpppG described above, as well as the ARCA 3′-OCH₃ and 2′-OCH₃ cap analogs (Jemielity, J. et al., RNA, 9: 1108-1122 (2003)). Additional cap analogs for use in embodiments of the invention include N7-benzylated dinucleoside tetraphosphate analogs (described in Grudzien, E. et al., RNA, 10: 1479-1487 (2004)), phosphorothioate cap analogs (described in Grudzien-Nogalska, E., et al., RNA, 13: 1745-1755 (2007)), and cap analogs (including biotinylated cap analogs) described in U.S. Pat. Nos. 8,093,367 and 8,304,529, incorporated by reference herein.

### Tail Structure

Typically, the presence of a “tail” serves to protect the mRNA from exonuclease degradation. The poly A tail is thought to stabilize natural messengers and synthetic sense RNA. Therefore, in certain embodiments a long poly A tail can be added to an mRNA molecule thus rendering the RNA more stable. Poly A tails can be added using a variety of art-recognized techniques. For example, long poly A tails can be added to synthetic or in vitro transcribed RNA using poly A polymerase (Yokoe, et al. Nature Biotechnology. 1996; 14:1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

In some embodiments, mRNAs include a 3′ poly(A) tail structure. Typically, the length of the poly A tail can be at least about 10, 50, 100, 200, 300, 400 at least 500 nucleotides. In some embodiments, a poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the length of the poly A or poly C tail is adjusted to control the stability of a modified sense mRNA molecule of the invention and, thus, the transcription of protein. For example, since the length of the poly A tail can influence the half-life of a sense mRNA molecule, the length of the poly A tail can be adjusted to modify the level of resistance of the mRNA to nucleases and thereby control the time course of polynucleotide expression and/or polypeptide production in a target cell.

### 5′ and 3′ Untranslated Region

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

### Delivery Vehicles

According to the present invention, reverse gene therapy (RGT) (e.g., a mRNA gene transfer construct comprising a reverse complement sequence encoding a protein of interest and a sequence encoding a human L1 retro-element) as described herein may be delivered as naked RNA (unpackaged) or via delivery vehicles. As used herein, the terms “delivery vehicle,” “transfer vehicle,” “nanoparticle” or grammatical equivalent, are used interchangeably.

In some embodiments, the RGT components (e.g., a mRNA gene transfer construct comprising a reverse complement sequence encoding a protein of interest and/or a sequence encoding a human L1 retro-element) may be delivered via a single delivery vehicle. In some embodiments, the RGT components may be delivered via one or more delivery vehicles each of a different composition.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags.

In some embodiments, the RGT delivery vehicle is a lipid nanoparticle (LNP). In some embodiments, the RGT delivery vehicle comprises a cell penetrating peptide (e.g., arginine-rich peptides such as HIV TAT). In some embodiments, the RGT delivery vehicle is a LNP comprising a cell penetrating peptide.

In some embodiments, the RGT delivery vehicle comprises a targeting agent (e.g., an antibody to a cell surface receptor). In some embodiments, the RGT delivery vehicle is a LNP comprising a targeting agent (e.g., an antibody to a cell surface receptor). In some embodiments, the targeting agent can achieve selective uptake by cells of interest for a particular therapeutic objective.

In some embodiments, the RGT delivery vehicle is an endosome. In some embodiments, the RGT delivery vehicle comprises an endosomal release agent to facilitate release of the polynucleotide (e.g, the mRNA gene transfer construct comprising a reverse complement sequence encoding a protein of interest and/or the sequence encoding a human L1 retro-element) from endosomal compartments. As used herein, “endosomal release agents” include any compound or peptide sequence that facilitates cargo exit from the endosome. Exemplary endosomal release agents include imidazoles, poly or oligoimidazoles, PEIs, peptides, fusogenic peptides, polycarboxylates, polycations, masked oligo or poly cations or anions, acetals, polyacetals, ketals/polyketyals, orthoesters, polymers with masked or unmasked cationic or anionic charges, amphiphilic block copolymers and dendrimers with masked or unmasked cationic or anionic charges. In some embodiments, the endosomal release agent is adapted from viral elements that promote escape from the endosome and deliver polynucleotides intact into the nucleus. In some embodiments, endosomal release agents include a hydrophobic membrane translocation sequence.

In some embodiments, the RGT delivery vehicle is not a viral vector. In some embodiments, RGT does not have the delivery vehicle packaging constraints that AAV gene therapy technologies suffer from (max size ˜4.5 Kb). Most therapeutic genes for RGT therapies are <15 Kb. RGT manufacturing uses cell-free, biosynthetic transcription technologies which are not limited by the size of the DNA template.

### Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue. In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. In some embodiments, a liposome comprises no more than three distinct lipid components. In some embodiments, one distinct lipid component is a sterol-based cationic lipid.

### Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

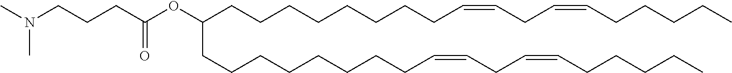 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

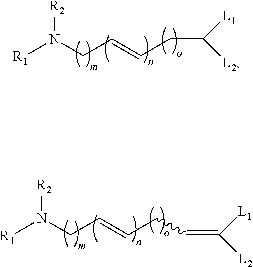

or a pharmaceutically acceptable salt thereof, wherein R1 and R2 are each independently selected from the group consisting of hydrogen, an optionally substituted, variably saturated or unsaturated C1-C20 alkyl and an optionally substituted, variably saturated or unsaturated C6-C20 acyl; wherein L1 and L2 are each independently selected from the group consisting of hydrogen, an optionally substituted C1-C30 alkyl, an optionally substituted variably unsaturated C1-C30 alkenyl, and an optionally substituted C1-C30 alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

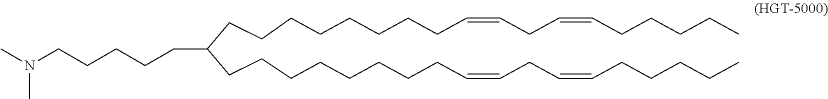 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

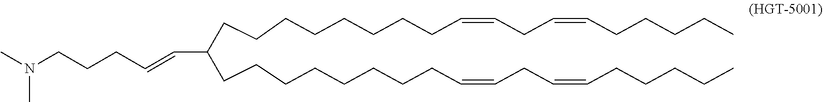 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

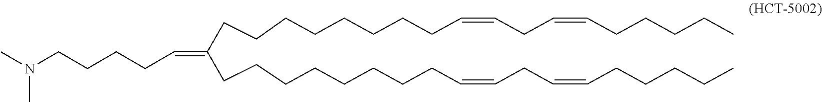 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

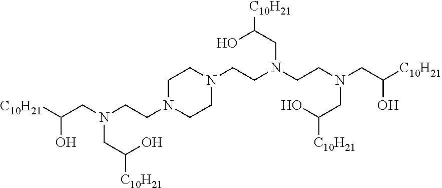

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

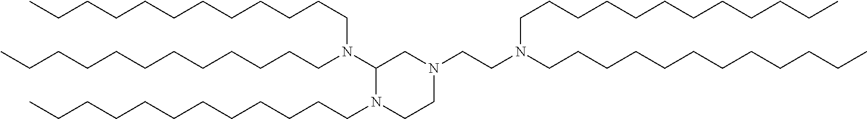 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

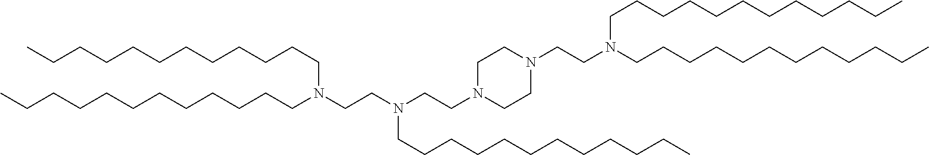 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

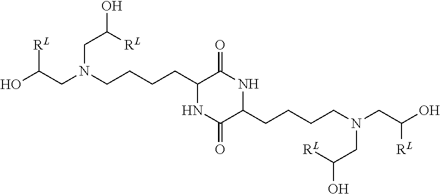

or pharmaceutically acceptable salts thereof, wherein each instance of RL is independently optionally substituted C6-C40 alkenyl. In certain embodiments, the compositions and methods of the present invention include a cationic lipid having a compound structure of:

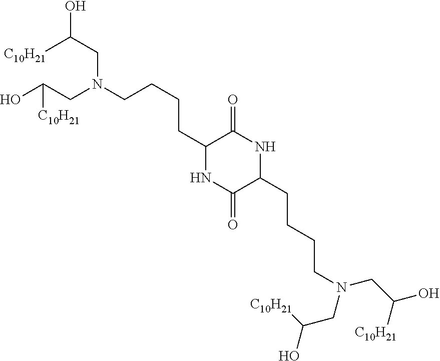

and pharmaceutically acceptable salts thereof. In certain embodiments, the compositions and methods of the present invention include a cationic lipid having a compound structure of:

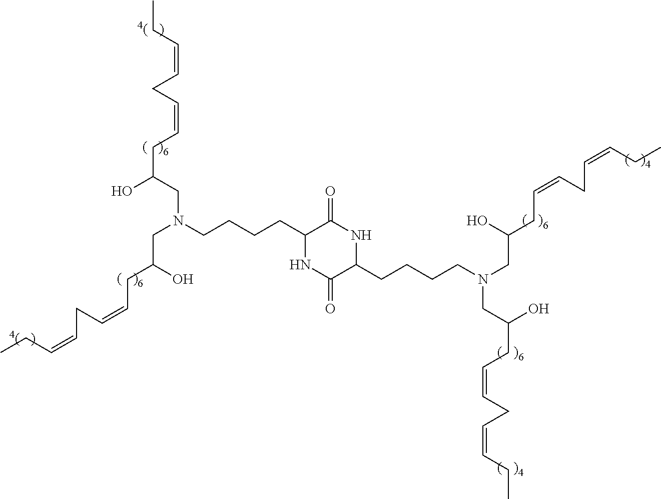

and pharmaceutically acceptable salts thereof. In certain embodiments, the compositions and methods of the present invention include a cationic lipid having a compound structure of:

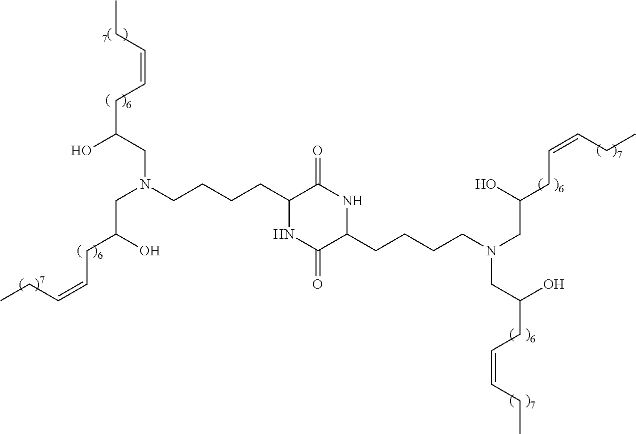

and pharmaceutically acceptable salts thereof. In certain embodiments, the compositions and methods of the present invention include a cationic lipid having a compound structure of:

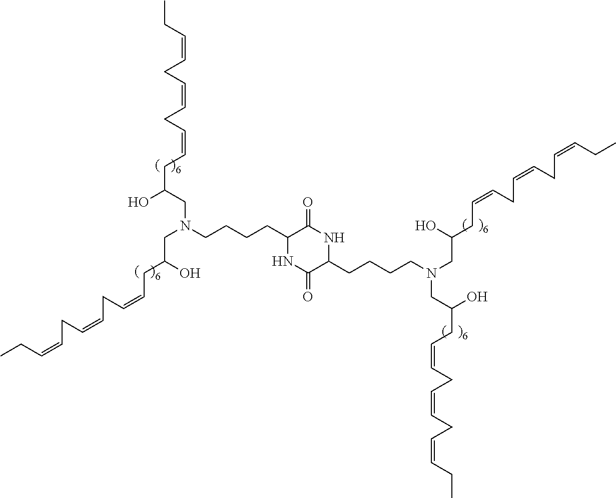

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

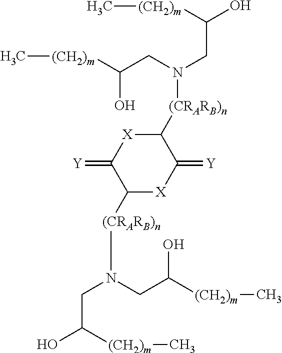

or a pharmaceutically acceptable salt thereof, wherein each X independently is O or S; each Y independently is O or S; each m independently is 0 to 20; each n independently is 1 to 6; each RA is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

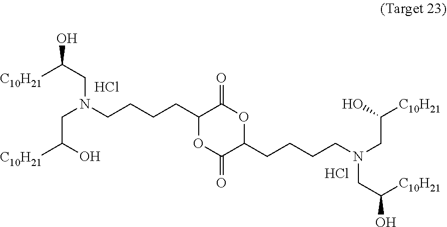

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

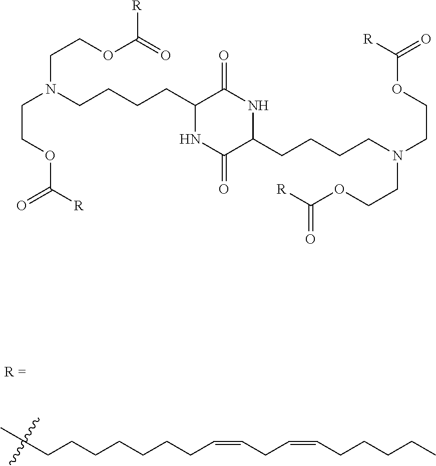

or a pharmaceutically acceptable salt thereof. In some embodiments, the compositions and methods of the present invention include a cationic lipid having the compound structure:

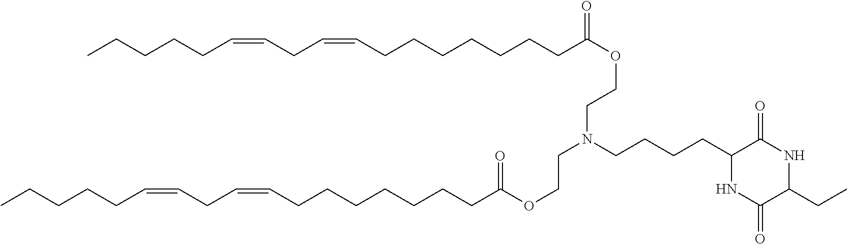 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

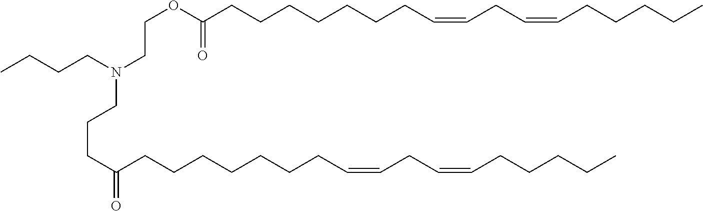 [SMILES: CCCCC/C=C\C/C=C\CCCCCCCC(=O)CCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

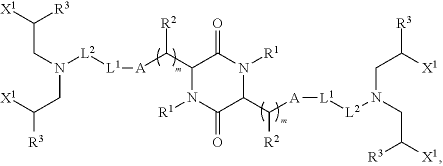

or a pharmaceutically acceptable salt thereof, wherein each R1 and R2 is independently H or C1-C6 aliphatic; each m is independently an integer having a value of 1 to 4; each A is independently a covalent bond or arylene; each L1 is independently an ester, thioester, disulfide, or anhydride group; each L2 is independently C2-C10 aliphatic; each X1 is independently H or OH; and each R3 is independently C6-C20 aliphatic. In some embodiments, the compositions and methods of the present invention include a cationic lipid of the following formula:

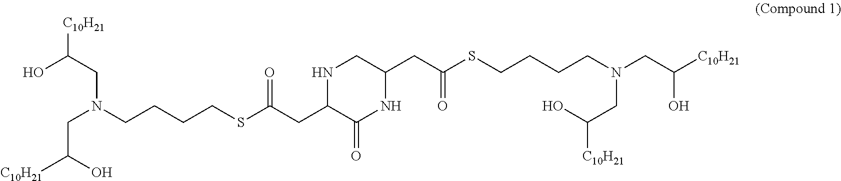

or a pharmaceutically acceptable salt thereof. In some embodiments, the compositions and methods of the present invention include a cationic lipid of the following formula:

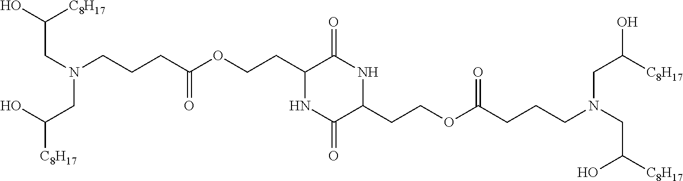

or a pharmaceutically acceptable salt thereof. In some embodiments, the compositions and methods of the present invention include a cationic lipid of the following formula:

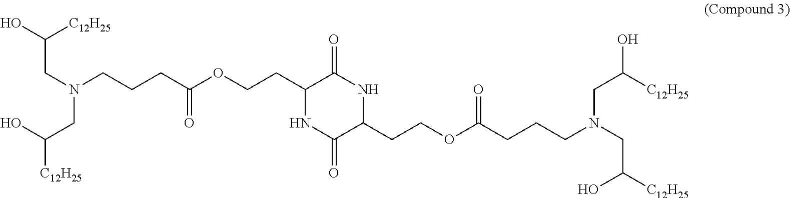

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

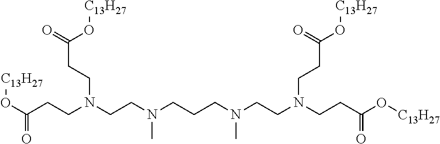

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

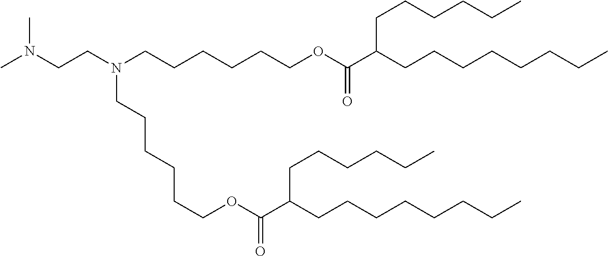 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

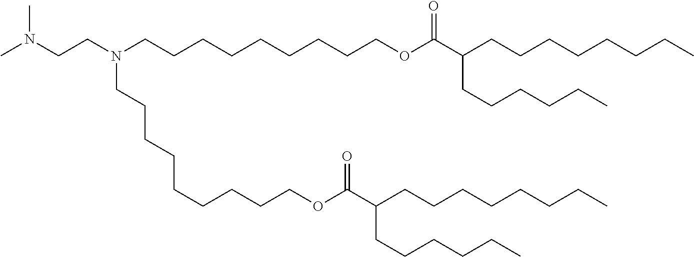 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

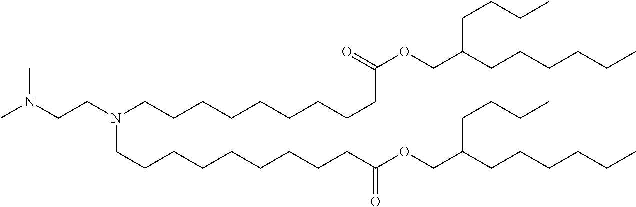 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

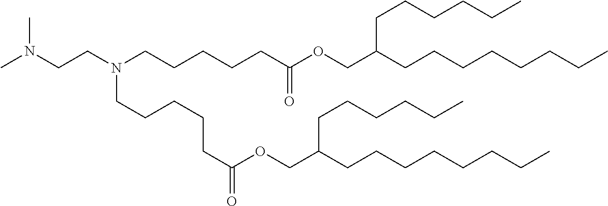 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

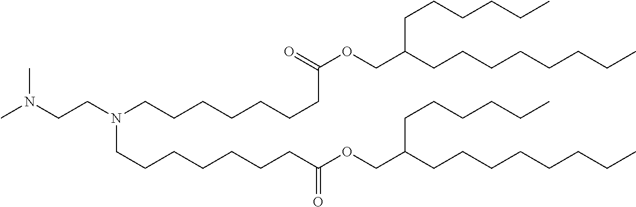 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

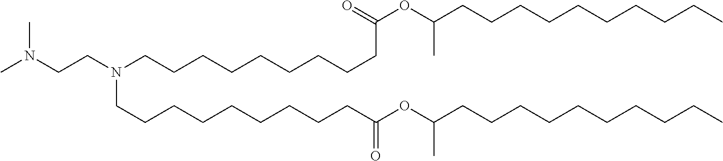 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

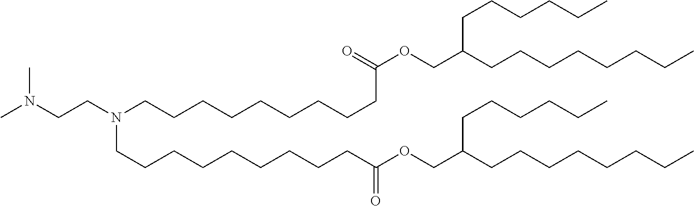 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

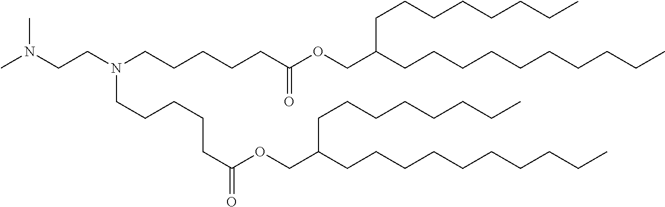 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

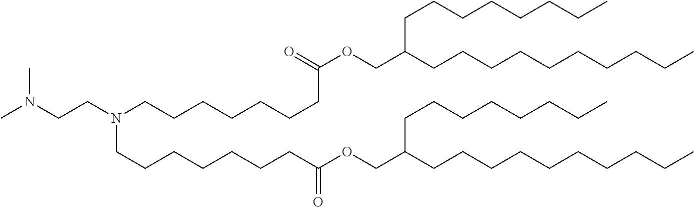 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

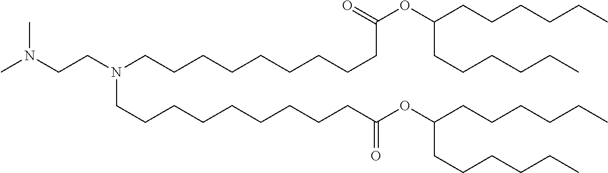 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

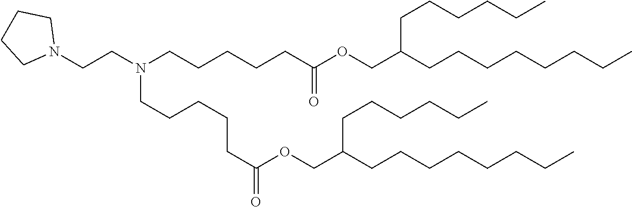 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

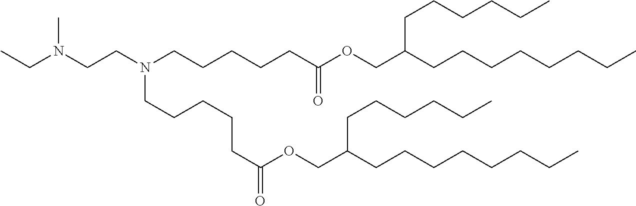 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

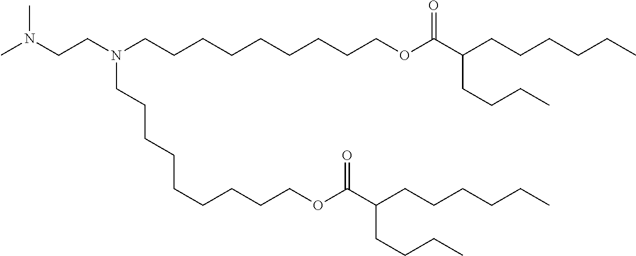 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

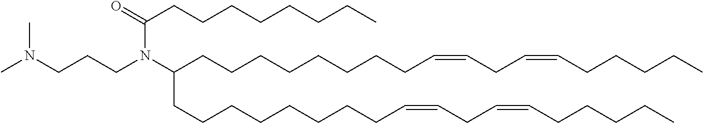 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

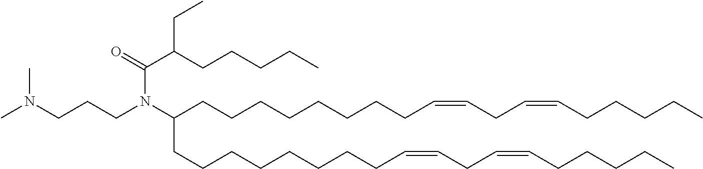 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

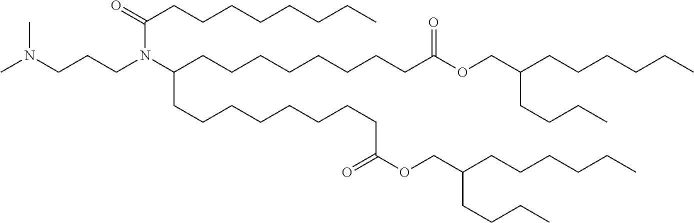 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

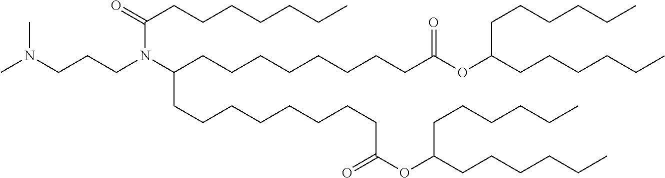 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

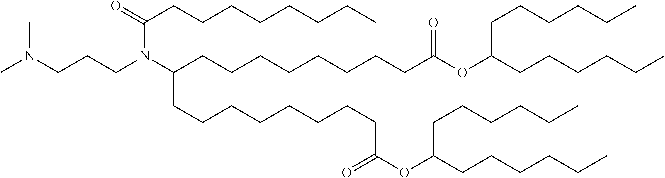 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

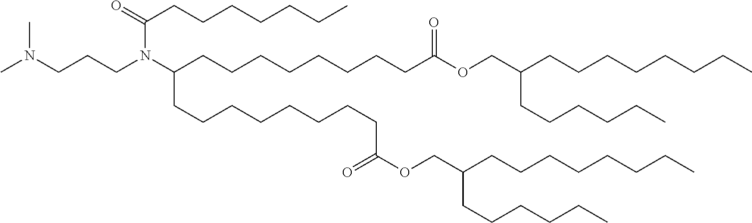 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

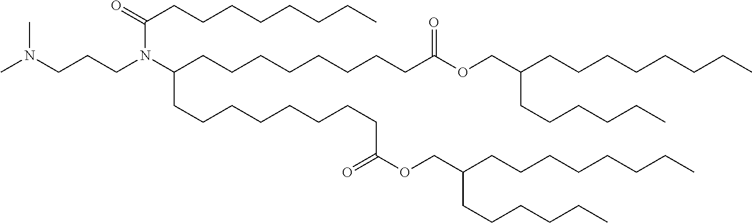 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

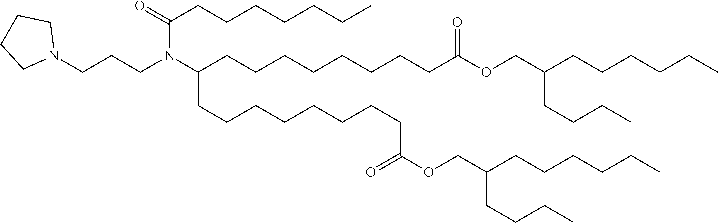 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

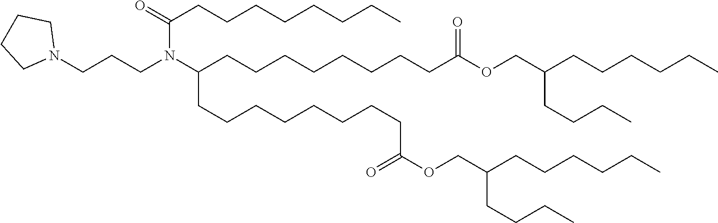 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

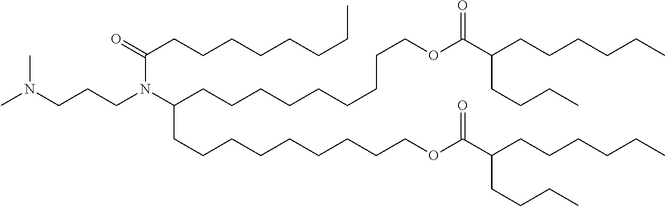 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

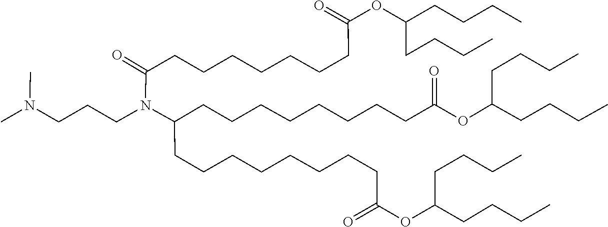 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

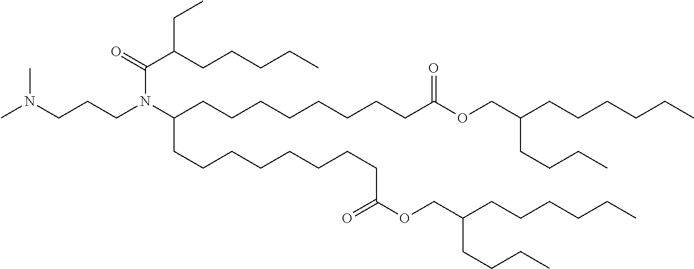 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

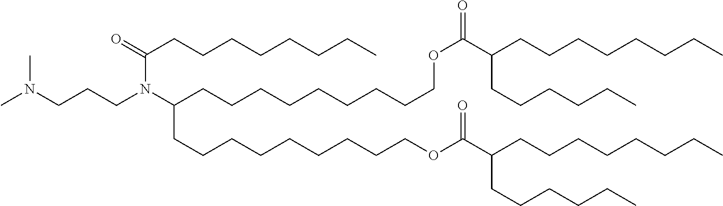 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

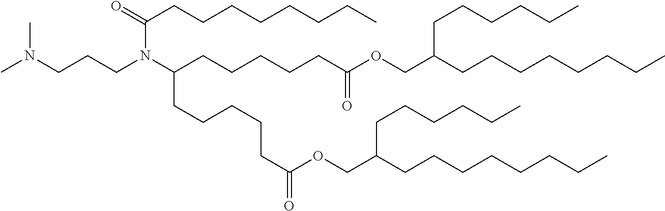 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

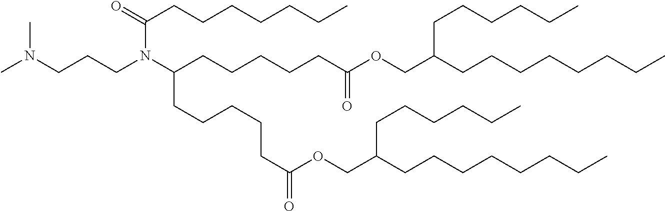 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

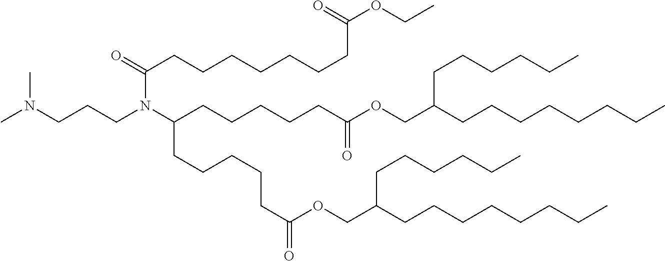 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

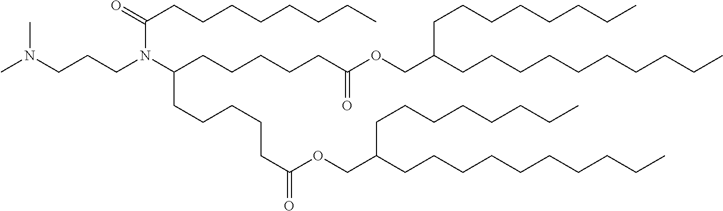 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

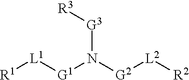

or a pharmaceutically acceptable salt thereof, wherein one of L1 or L2 is —O(C═O)—, —(C═O)O—, —C(═O)—, —O—, —S(O)x, —S—S—, —C(═O)S—, —SC(═O)—, —NRaC(═O)—, —C(═O)NRa—, NRaC(═O)NRa—, —OC(═O)NRa—, or —NRaC(═O)O—; and the other of L1 or L2 is —O(C═O)—, —(C═O)O—, —C(═O)—, —O—, —S(O) x, —S—S—, —C(═O)S—, SC(═O)—, —NRaC(═O)—, —C(═O)NRa-, NRaC(═O)NRa-, —OC(═O)NRa- or —NRaC(═O)O— or a direct bond; G1 and G2 are each independently unsubstituted C1-C12 alkylene or C1-C12 alkenylene; G3 is C1-C24 alkylene, C1-C24 alkenylene, C3-C8 cycloalkylene, C3-C8 cycloalkenylene; Ra is H or C1-C12 alkyl; R1 and R2 are each independently C6-C24 alkyl or C6-C24 alkenyl; R3 is H, OR5, CN, —C(═O)OR4, —OC(═O)R4 or —NR5 C(═O)R4; R4 is C1-C12 alkyl; R5 is H or C1-C6 alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

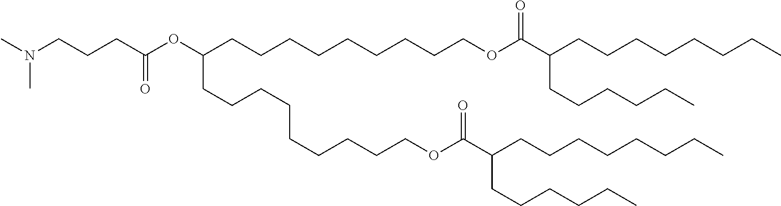 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

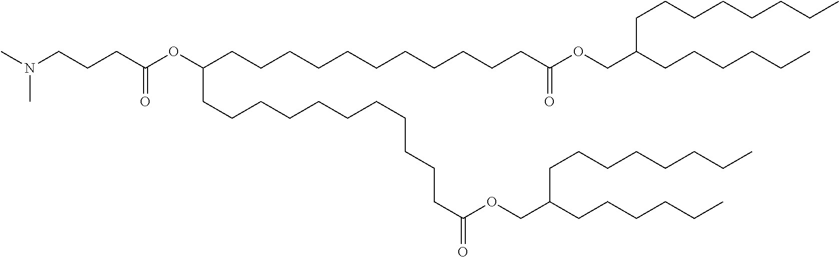 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

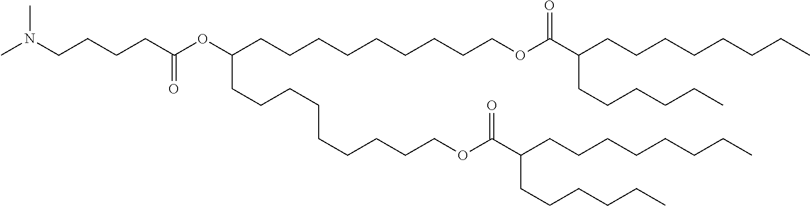 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

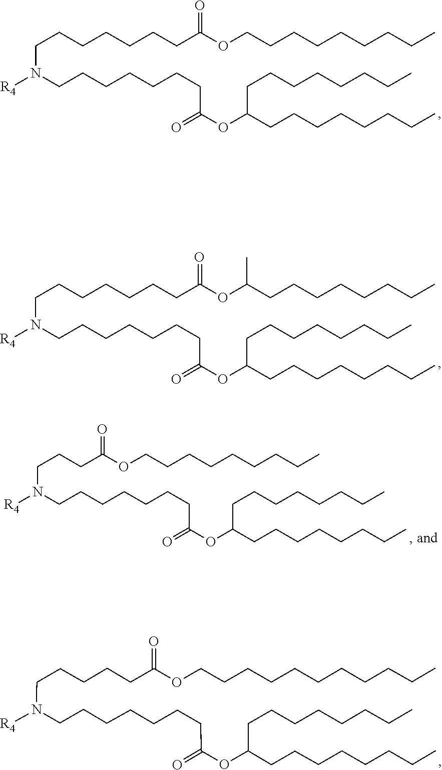

and pharmaceutically acceptable salts thereof. For any one of these four formulas, R4 is independently selected from —(CH2)nQ and —(CH2) nCHQR; Q is selected from the group consisting of —OR, —OH, —O(CH2)nN(R)2, —OC(O)R, —CX3, —CN, —N(R)C(O)R, —N(H)C(O)R, —N(R)S(O)2R, —N(H)S(O)2R, —N(R)C(O)N(R)2, —N(H)C(O)N(R)2, —N(H)C(O)N(H)(R), —N(R)C(S)N(R)2, —N(H)C(S)N(R)2, —N(H)C(S)N(H)(R), and a heterocycle; and n is 1, 2, or 3. In certain embodiments, the compositions and methods of the present invention include a cationic lipid having a compound structure of:

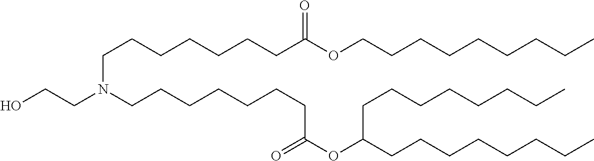 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

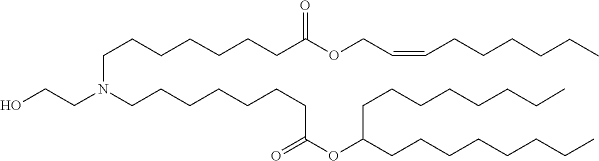 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

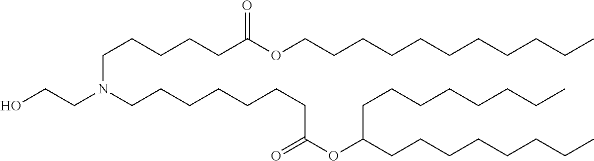 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

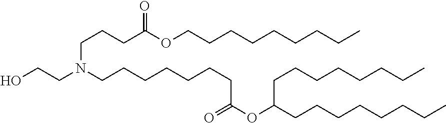 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

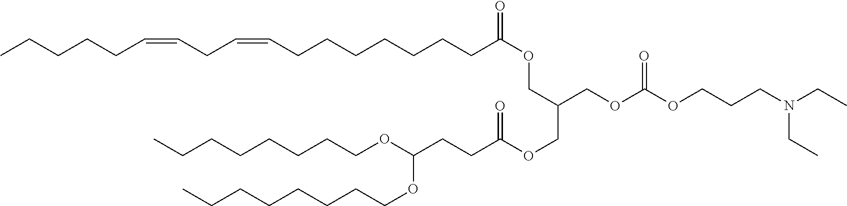 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

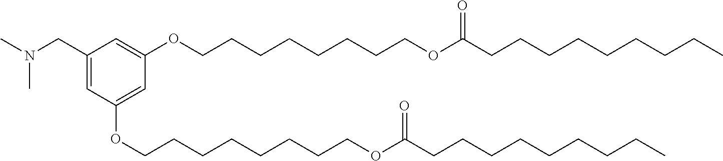 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

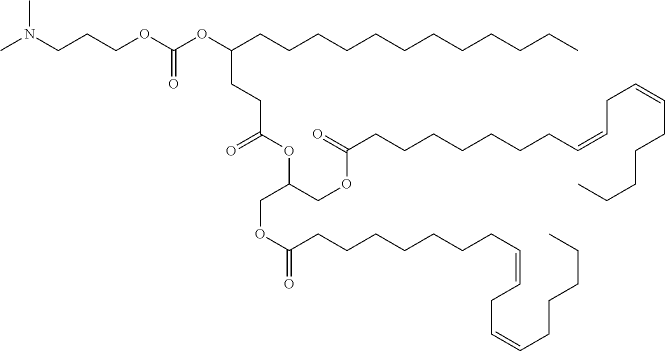 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

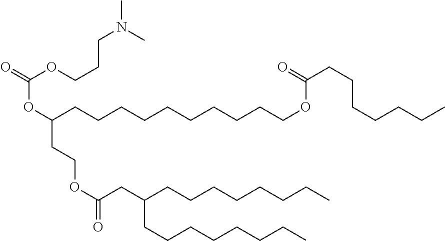 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

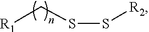

wherein R1 is selected from the group consisting of imidazole, guanidinium, amino, imine, enamine, an optionally-substituted alkyl amino (e.g., an alkyl amino such as dimethylamino) and pyridyl; wherein R2 is selected from the group consisting of one of the following two formulas:

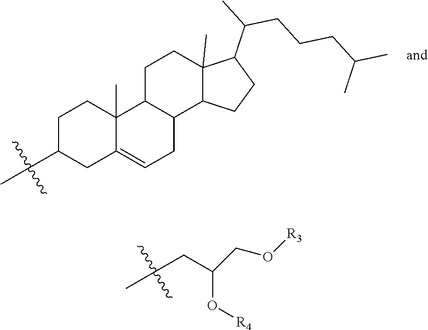

and wherein R3 and R4 are each independently selected from the group consisting of an optionally substituted, variably saturated or unsaturated C6-C20 alkyl and an optionally substituted, variably saturated or unsaturated C6-C20 acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

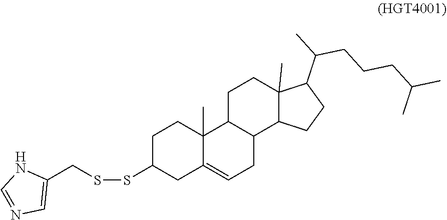 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” having a compound structure of:

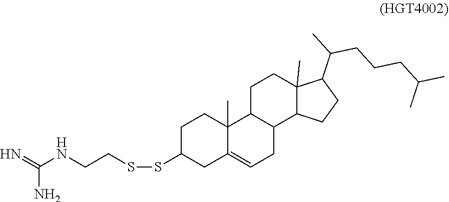 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

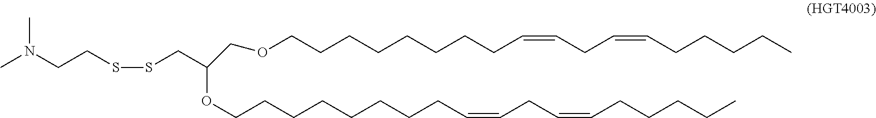 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

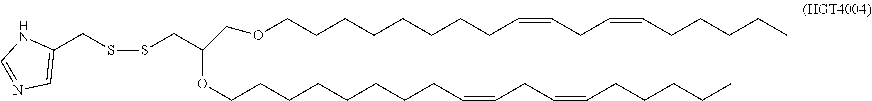 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

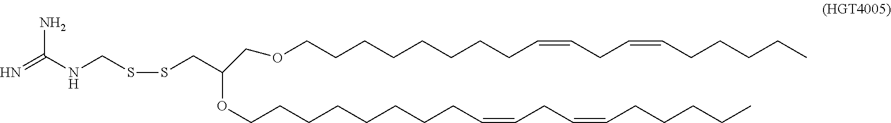 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in International Application No. PCT/US2019/032522, and incorporated herein by reference. In certain embodiments, the compositions and methods of the present invention include a cationic lipid that is any of general formulas or any of structures (1a)-(21a) and (1b)-(21b) and (22)-(237) described in International Application No. PCT/US2019/032522. In certain embodiments, the compositions and methods of the present invention include a cationic lipid that has a structure according to Formula (I′),

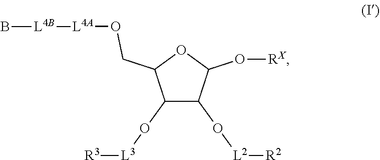

wherein:
RX is independently —H, -L1-R1, or -L5A-L5B-B′;
each of L1, L2, and L3 is independently a covalent bond, —C(O)—, —C(O)O—, —C(O)S—, or —C(O)NRL-;
each L4A and L5A is independently —C(O)—, —C(O)O—, or —C(O)NRL-;
each L4B and L5B is independently C1-C20 alkylene; C2-C20 alkenylene; or C2-C20 alkynylene;
each B and B′ is NR4R5 or a 5- to 10-membered nitrogen-containing heteroaryl;
each R1, R2, and R3 is independently C6-C30 alkyl, C6-C30 alkenyl, or C6-C30 alkynyl;
each R4 and R5 is independently hydrogen, C1-C10 alkyl; C2-C10 alkenyl; or C2-C10 alkynyl; and
each RL is independently hydrogen, C1-C20 alkyl, C2-C20 alkenyl, or C2-C20 alkynyl.

In certain embodiments, the compositions and methods of the present invention include a cationic lipid that is Compound (139) of International Application No. PCT/US2019/032522, having a compound structure of:

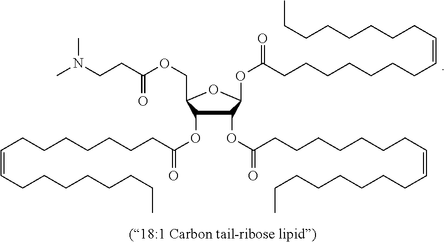

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9,12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9,12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9,12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

### Non-Cationic Helper Lipids

In some embodiments, the liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, a non-cationic lipid is a neutral lipid, i.e., a lipid that does not carry a net charge in the conditions under which the composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, the liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), which has the following structure,

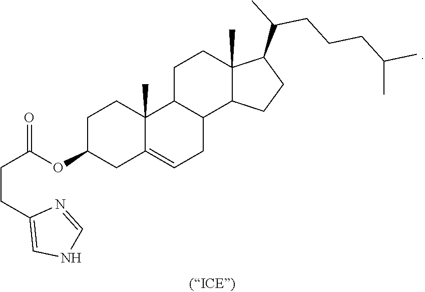 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

In some embodiments, the cholesterol-based lipid may comprise a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEG-Modified Lipids

In some embodiments, the liposome comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C6-C20 length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18).

The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio.

### Ratio of Distinct Lipid Components

A suitable liposome for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

$$( y + z ) = 100 - x .$$

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

### Exemplary Lipids for Nanoparticles

In some embodiments, the liposomal transfer vehicles for use in the compositions of the invention comprises one or more cationic lipids, one or more non-cationic lipids, one or more cholesterol-based lipids and one or more PEG-modified lipids.

In some embodiments, the liposomal transfer vehicles comprise one or more cationic lipids selected from the group consisting of C12-200, MC3, DLinDMA, DLinkC2DMA, cKK-E12, ICE (Imidazole-based), HGT5000, HGT5001, OF-02, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

In some embodiments, the liposomal transfer vehicles comprise one or more non-cationic lipids selected from the group consisting of DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (,2-dioleoyl-sn-glycero-3-phospho-(1′-rac-glycerol)) and combinations thereof.

In some embodiments, the liposomal transfer vehicles comprise one or more cholesterol-based lipids are cholesterol and/or PEGylated cholesterol.

### Formation of Liposomes

The liposomal transfer vehicles for use in the compositions of the invention can be prepared by various techniques which are presently known in the art. The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (Zeta potential), targeting ligands and/or combinations thereof. In some embodiments, the one or more liposome may have a different composition of cationic lipids, neutral lipid, PEG-modified lipid and/or combinations thereof. In some embodiments the one or more liposomes may have a different molar ratio of cationic lipid, neutral lipid, cholesterol and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

### Liposome Size

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues. For example, a liposome may be sized such that its dimensions are larger than the fenestrations of the endothelial layer lining hepatic sinusoids to thereby limit distribution of the liposomes to hepatocytes.

In some embodiments, the size of a liposome is determined by the length of the largest diameter of the liposome particle. In some embodiments, a suitable liposome has a size no greater than about 250 nm (e.g., no greater than about 225 nm, 200 nm, 175 nm, 150 nm, 125 nm, 100 nm, 75 nm, or 50 nm). In some embodiments, a suitable liposome has a size ranging from about 10-250 nm (e.g., ranging from about 10-225 nm, 10-200 nm, 10-175 nm, 10-150 nm, 10-125 nm, 10-100 nm, 10-75 nm, or 10-50 nm). In some embodiments, a suitable liposome has a size ranging from about 100-250 nm (e.g., ranging from about 100-225 nm, 100-200 nm, 100-175 nm, 100-150 nm). In some embodiments, a suitable liposome has a size ranging from about 10-100 nm (e.g., ranging from about 10-90 nm, 10-80 nm, 10-70 nm, 10-60 nm, or 10-50 nm). In a particular embodiment, a suitable liposome has a size less than about 100 nm.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Pharmaceutical Compositions and Methods

Messenger RNA therapy is an effective approach for the treatment of a variety of diseases. mRNA can be administered to a patient in need of the therapy for production of the protein encoded by the mRNA within the patient's body. Lipid nanoparticles are commonly used to encapsulate mRNA for efficient in vivo delivery of mRNA.

To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

Provided liposomally-encapsulated or associated mRNAs, and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Alternatively or additionally, liposomally encapsulated mRNAs and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

The methods and compositions of the present invention may be used to preferentially target a vast number of target cells. For example, contemplated target cells include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Provided methods of the present invention contemplate single as well as multiple administrations of a therapeutically effective amount of the therapeutic agents (e.g., mRNA encoding a protein of interest) described herein. Therapeutic agents can be administered at regular intervals, depending on the nature, severity and extent of the subject's condition. In some embodiments, a therapeutically effective amount of the therapeutic agents (e.g., mRNA encoding a protein of interest) of the present invention may be administered intrathecally periodically at regular intervals (e.g., once every year, once every six months, once every five months, once every three months, bimonthly (once every two months), monthly (once every month), biweekly (once every two weeks), twice a month, once every 30 days, once every 28 days, once every 14 days, once every 10 days, once every 7 days, weekly, twice a week, daily or continuously).

In some embodiments, provided liposomes and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice a day, daily or every other day. In some embodiments, the compositions of the present invention are administered to a subject as a single dose of RGT (e.g., a nanoparticle comprising a mRNA gene transfer construct comprising a reverse complement sequence encoding a protein of interest; and a sequence encoding a human L1 retro-element). In some embodiments, RGT is administered annually (e.g., once a year, every 12 months, every 24 months, every 36 months). In some embodiments, RGT is administered every 2 years, every 3 years, every 4 years, or every 5 years. In some embodiments, RGT is administered as needed.

Also contemplated are compositions and liposomes which are formulated for depot administration (e.g., intramuscularly, subcutaneously, intravitreally) to either deliver or release a mRNA over extended periods of time. Preferably, the extended-release means employed are combined with modifications made to the mRNA to enhance stability.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating a disease). For example, a therapeutically effective amount may be an amount sufficient to achieve a desired therapeutic and/or prophylactic effect. Generally, the amount of a therapeutic agent (e.g., mRNA encoding a protein of interest) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; and like factors as is well known in the medical arts.

Also contemplated herein are lyophilized pharmaceutical compositions comprising one or more of the liposomes disclosed herein and related methods for the use of such compositions as disclosed for example, in International Patent Application PCT/US12/41663, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual's bodily fluids.

Provided liposomes and compositions may be administered to any desired tissue. In some embodiments, the mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

According to various embodiments, the method for gene delivery comprises contacting a cell with the mRNA delivery vehicle (e.g., a nanoparticle comprising RGT) in vivo. In some embodiments, contacting of the cell with the mRNA delivery vehicle (e.g., a nanoparticle comprising RGT) is carried out ex vivo. In some embodiments, contacting the cell with the mRNA delivery vehicle (e.g., a nanoparticle comprising RGT) is carried out in vitro.

In some embodiments, the cell is a stem cell, a hematopoietic precursor cell, a granulocyte, a mast cell, a monocyte, a dendritic cell, a B cell, a T cell, a natural killer cell, a fibroblast, a muscle cell, a cardiac cell, a hepatocyte, a lung progenitor cell, or a neuronal cell. In some embodiments, the cell is a T cell. In some embodiments, the mRNA encodes a protein that is capable of modulating an immune response in an individual in which it is expressed. In some embodiments, the mRNA delivery vehicle (e.g., a nanoparticle comprising RGT) comprises an mRNA encoding a therapeutic protein.

In some embodiments, the present invention provides a method of gene delivery for a disease in an individual comprising administering to the individual an effective amount of a pharmaceutical composition according to any of the embodiments described above. In some embodiments, the pharmaceutical composition is administered via intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration. In some embodiments, the pharmaceutical composition is administered via injection into a blood vessel wall or tissue surrounding the blood vessel wall. In some embodiments, the injection is through a catheter with a needle.

In some embodiments, according to any of the methods of treating a disease described above, the disease is selected from the group consisting of cancer, diabetes, autoimmune diseases, hematological diseases, cardiac diseases, vascular diseases, inflammatory-diseases, fibrotic diseases, viral infectious diseases, hereditary diseases, ocular diseases, liver diseases, lung diseases, muscle diseases, protein deficiency diseases, lysosomal storage diseases, neurological diseases, kidney diseases, aging and degenerative diseases, and diseases characterized by cholesterol level abnormality. In some embodiments, the disease is selected from the group consisting metabolic disease (e.g. NASH), central nervous system diseases, peripheral nervous system diseases, muscle diseases.

In some embodiments, the disease is a metabolic disease (e.g. Non-alcoholic steatohepatitis (NASH)). In some embodiments, the pharmaceutical composition comprises an mRNA gene transfer construct comprising a reverse complement sequence encoding a protein of interest; and a sequence encoding a human L1 retro-element. In some embodiments, the mRNA encodes a protein useful for treating the metabolic disease. In some embodiments, the disease is characterized by an abnormal protein. In some embodiments, the pharmaceutical composition comprises a mRNA encoding a functional variant of the non-functional protein contributing to the disease.

In some embodiments, the disease is a central nervous system disease. In some embodiments, the pharmaceutical composition comprises an mRNA gene transfer construct comprising a reverse complement sequence encoding a protein of interest; and a sequence encoding a human L1 retro-element. In some embodiments, the mRNA encodes a protein useful for treating the central nervous system disease. In some embodiments, the disease is characterized by an abnormal protein. In some embodiments, the pharmaceutical composition comprises a mRNA encoding a functional variant of the non-functional protein contributing to the disease.

In some embodiments, the disease is a peripheral nervous system disease. In some embodiments, the pharmaceutical composition comprises an mRNA gene transfer construct comprising a reverse complement sequence encoding a protein of interest; and a sequence encoding a human L1 retro-element. In some embodiments, the mRNA encodes a protein useful for treating the peripheral nervous system disease. In some embodiments, the disease is characterized by an abnormal protein. In some embodiments, the pharmaceutical composition comprises a mRNA encoding a functional variant of the non-functional protein contributing to the disease.

In some embodiments, the disease is a muscle disease. In some embodiments, the pharmaceutical composition comprises an mRNA gene transfer construct comprising a reverse complement sequence encoding a protein of interest; and a sequence encoding a human L1 retro-element. In some embodiments, the mRNA encodes a protein useful for treating the muscle disease. In some embodiments, the disease is characterized by an abnormal protein. In some embodiments, the pharmaceutical composition comprises a mRNA encoding a functional variant of the non-functional protein contributing to the disease.

In some embodiments, the disease is a protein deficiency disease. In some embodiments, the pharmaceutical composition comprises an mRNA gene transfer construct comprising a reverse complement sequence encoding a protein of interest; and a sequence encoding a human L1 retro-element. In some embodiments, the mRNA encodes a deficient protein contributing to the disease. In some embodiments, the disease is characterized by an abnormal protein. In some embodiments, the pharmaceutical composition comprises a mRNA encoding a functional variant of the non-functional protein contributing to the disease.

In some embodiments, the disease is cancer. In some embodiments, the cancer is a solid tumor, and the pharmaceutical composition comprises an mRNA gene transfer construct comprising a reverse complement sequence encoding a tumor suppressor protein useful for treating the solid tumor. In some embodiments, the cancer is cancer of the liver, lung, kidney, colorectum, or pancreas. In some embodiments, the cancer is a hematological malignancy, and the pharmaceutical composition comprises an mRNA gene transfer construct comprising a reverse complement sequence encoding a tumor suppressor protein useful for treating the hematological malignancy.

In some embodiments, RGT is used in immunotherapy. In some embodiments, immunotherapy refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies (e.g., adoptive T cell therapy, tumor-infiltrating lymphocyte (TIL) immunotherapy, autologous cell therapy, autologous cell therapy, and allogeneic T cell transplantation). In some embodiments, RGT is administered using ex vivo gene transfer to T cells, induced pluripotent stem cells, stem cells, bone marrow stem cells or other blood cells.

In some embodiments, the pharmaceutical composition comprises an mRNA gene transfer construct comprising a sequence encoding a chimeric antigen receptor (CAR) or an engineered T cell receptor (TCR). In some embodiments, RGT comprising a sequence encoding a CAR or TCR is administered ex vivo for T cell therapies. In some embodiments, RGT comprising a sequence encoding a CAR or TCR is administered in vivo. In some embodiments, according to any of the methods of treating a disease described above, the disease is a viral infection disease, and the pharmaceutical composition comprises an mRNA encoding a protein involved in the viral infectious disease development and/or progression.

In some embodiments, according to any of the methods of treating a disease described above, the disease is a hereditary disease, and the pharmaceutical composition comprises an mRNA gene transfer construct comprising a reverse complement sequence encoding one or more proteins involved in the hereditary disease development and/or progression.

In some embodiments, according to any of the methods of treating a disease described above, the disease is an aging or degenerative disease, and the pharmaceutical composition comprises an mRNA gene transfer construct comprising a reverse complement sequence encoding one or more proteins involved in the aging or degenerative disease development and/or progression.

In some embodiments, according to any of the methods of treating a disease described above, the disease is a fibrotic or inflammatory disease, and the pharmaceutical composition comprises an mRNA gene transfer construct comprising a reverse complement sequence encoding one or more proteins involved in the fibrotic or inflammatory disease development and/or progression.

In some embodiments, according to any of the methods of treating a disease described above, the individual is human.

In some embodiments, the present invention provides a kit comprising a composition comprising RGT according to any of the embodiments described above.

### EXAMPLES

While certain compounds, compositions and methods of the present invention have been described with specificity in accordance with certain embodiments, the following examples serve only to illustrate the compounds of the invention and are not intended to limit the same.

### Example 1. Exemplary XE Sequences for Increased Transgene Expression

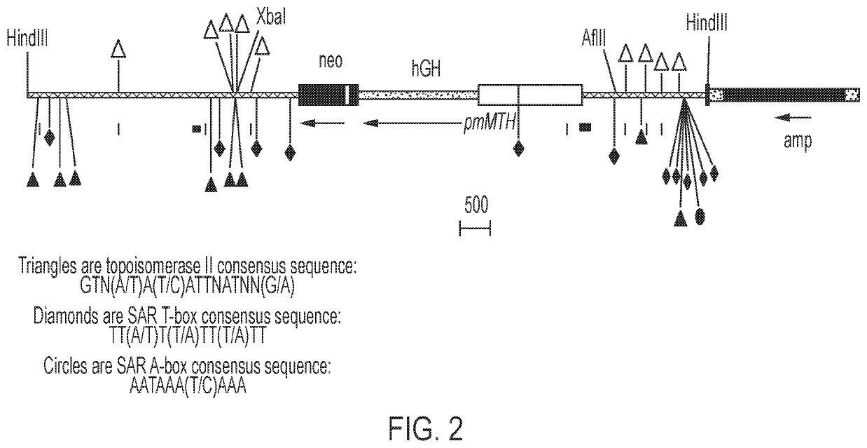

This example provides exemplary constructs including XE sequences that increase transgene expression (FIG. 2). hGH embedded within XE sequences derived from the hypoxanthine-guanine phosphoribosyltransferase (HGPRT) gene stimulated stable expression as high as 10 fold over hGH expression observed in the absence of these sequences with pXGH5 or pXGH301. The HGPRT1 gene contains several copies of consensus sequences for nuclear scaffold associated regions (SAR)/matrix attachment regions (MAR). hGH expression was evaluated in nearly 100 independent cell strains transfected with pE3neohGH11 (approximately equal numbers of either supercoiled or linearized). Primary human fibroblasts were transfected with HindIII digested (linearized) pE3neohGH11 (HF165) or supercoiled pE3NEOhGH11 (HFl 72) by electroporation at 250 volts and 100 μg of DNA. Colonies were isolated approximately 14 days following transfection and selection in 0.8 mg/ml G418. Standard schedules for the propogation and hGH expression analysis of cell strains were followed in these experiments. 100% of strains transferred to 96 well plates were expressing hGH. hGH expression data for linearized or supercoiled pE3neohGH11 is shown in Tables 1 and 2, respectively. Examination of the hGH levels shown in Table 1 show expression of greater than 30 μg per million cells per day, compared with plasmids containing the same promoter (mMTI) in the absence of HGPRT sequences (Table 3).

TABLE 1
Expression of hGH from pE3NEOhGH11

| | | ng/1e6 | Estimated copy number |
|---|---|---|---|
| CELL | STRAIN | CELLS/24 h | per haploid genome |
| HF165 | 24 | 2353630.34 | 100 |
| HF165 | 26 | 139920.28 | 8 |
| HF165 | 40 | 73465.63 | 10 |
| HF165 | 3 | 72512.87 | 50 |
| HF165 | 22 | 70451.48 | 20 |
| HF165 | 27 | 69057.47 | 8 |
| HF165 | 31 | 57876.89 | 8 |
| HF165 | 21 | 56379.36 | 20 |
| HF165 | 18 | 47888.22 | 10 |
| HF165 | 37 | 43233.73 | 15 |
| HF165 | 23 | 42905.08 | 8 |
| HF165 | 1 | 39296.76 | nd |
| HF165 | 25 | 36933.52 | 8 |
| HF165 | 8 | 35663.21 | 8 |
| HF165 | 6 | 35088.17 | 8 |
| HF165 | 12 | 34387.48 | 20 |
| HF165 | 17 | 33048.08 | 8 |
| HF165 | 32 | 29334.51 | 15 |
| HF165 | 28 | 29179.22 | 3 |
| HF165 | 7 | 28260.42 | 4 |
| HF165 | 35 | 25876.59 | 3 |
| HF165 | 2 | 23026.13 | 4 |
| HF165 | 30 | 22955.88 | 20 |
| HF165 | 9 | 20557.21 | 6 |
| HF165 | 15 | 20506.00 | 5 |
| HF165 | 13 | 19121.13 | 10 |
| HF165 | 36 | 18951.54 | 8 |
| HF165 | 11 | 15789.24 | 2 |
| HF165 | 20 | 14369.20 | 4 |
| HF165 | 29 | 14000.46 | 100 |
| HF165 | 34 | 13348.28 | 6 |
| HF165 | 14 | 12732.59 | 20 |
| HF165 | 10 | 11650.76 | 4 |
| HF165 | 39 | 10157.01 | 8 |
| HF165 | 33 | 9272.54 | 2 |
| HF165 | 5 | 7968.68 | 3 |
| HF165 | 38 | 6469.76 | 3 |
| HF165 | 4 | 6397.47 | 1 |
| HF165 | 19 | 6197.21 | nd |
| HF165 | 16 | 179.09 | nd |
| | Mean | 37255.61 | 15 |
| | Minimum | 179.09 | 1 |
| | Maximum | 235360.34 | 100 |
| | Number | 40.00 | 37 |

TABLE 2
hGh Expression from pE3neohGH11 - Supercoiled DNA

| | | Cell# | Ng/1e6 | Estimated Copy number |
|---|---|---|---|---|
| CELL | STRAIN | ×1e6 | CELLS/24 h | per haploid genome |
| HF172 | 2 | 1.66 | 52923 | 100 |
| HF172 | 32 | 1.11 | 37327 | 20 |
| HF172 | 27 | 2.67 | 26614 | 100 |
| HF172 | 17 | 1.95 | 26251 | 50 |
| HF172 | 10 | 2.11 | 15873 | 20 |
| HF172 | 30 | 1.72 | 14202 | 15 |
| HF172 | 1 | 0.33 | 10232 | 10 |
| HF172 | 54 | 1.79 | 7705 | 1 |
| HF172 | 40 | 1.29 | 7491 | 10 |
| HF172 | 52 | 2.90 | 7329 | 10 |
| HF172 | 33 | 2.03 | 6780 | 10 |
| HF172 | 50 | 5.44 | 6358 | 4 |
| HF172 | 47 | 1.58 | 5262 | no dna |
| HF172 | 12 | 1.78 | 5153 | 1 |
| HF172 | 31 | 2.60 | 5134 | 10 |
| HF172 | 5 | 0.85 | 4903 | 2 |
| HF172 | 26 | 1.44 | 4881 | 1 |
| HF172 | 9 | 0.37 | 4644 | 10 |
| HF172 | 53 | 0.87 | 4577 | 4 |
| HF172 | 29 | 0.77 | 4437 | 2 |
| HF172 | 45 | 0.50 | 4197 | 1 |
| HF172 | 18 | 1.48 | 4168 | 20 |
| HF172 | 13 | 1.77 | 3742 | 2 |
| HF172 | 3 | 4.86 | 3569 | 5 |
| HF172 | 11 | 0.73 | 3267 | 1 |
| HF172 | 7 | 1.70 | 2813 | 1 |
| HF172 | 42 | 1.23 | 2783 | 2 |
| HF172 | 22 | 2.76 | 2767 | 1 |
| HF172 | 48 | 1.25 | 2594 | 5 |
| HF172 | 49 | 2.64 | 2581 | 1 |
| HF172 | 37 | 1.09 | 2570 | 2 |
| HF172 | 44 | 1.25 | 2289 | 1 |
| HF172 | 19 | 2.12 | 2235 | 5 |
| HF172 | 51 | 1.43 | 2213 | 1 |
| HF172 | 41 | 2.22 | 2156 | 2 |
| HF172 | 14 | 2.09 | 2125 | 1 |
| HF172 | 39 | 1.79 | 2079 | 1 |
| HF172 | 6 | 1.16 | 1976 | 1 |
| HF172 | 25 | 0.45 | 1775 | 1 |
| HF172 | 28 | 2.94 | 1730 | 1 |
| HF172 | 34 | 0.78 | 1611 | 1 |
| HF172 | 21 | 1.80 | 1323 | 1 |
| HF172 | 15 | 2.07 | 1302 | 1 |
| HF172 | 36 | 0.87 | 1117 | 1 |
| HF172 | 46 | 1.16 | 876 | 2 |
| HF172 | 8 | 1.26 | 588 | 30 |
| HF172 | 38 | 1.01 | 403 | 5 |
| HF172 | 24 | 2.96 | 316 | 1 |
| HF172 | 16 | 1.72 | 168 | 1 |
| HF172 | 20 | 0.58 | 92 | 1 |
| HF172 | 23 | 3.90 | 54 | 0 |
| HF172 | 43 | 1.20 | 43 | 1 |
| HF172 | 4 | 1.95 | 7 | 1 |
| HF172 | 35 | 1.72 | 1 | 0 |
| | Mean | 1.74 | 5918.62 | 9 |
| | Maximum | 5.44 | 52923.17 | 100 |
| | Minimum | 0.33 | 1.35 | 0 |
| | n- | 54 | 54 | 53 |

Average expression was between 20-37 μg/million cells/day. This was nearly 10 times the frequency seen with co-transfection of pCDNEO and pXGH5 or pXGH301. Previous experiments demonstrated that the isolation and study of approximately 300 strains with pXGH5 or pXGH301 (the same hGH expression unit) was required to obtain 5 strains making greater than 20 μg per day. The highest known levels of hGH was produced in primary human fibroblasts (HF165-24) at 235 μg/million cells/day. The only difference between these experiments and previous transfections was the flanking HGPRT sequence.

TABLE 3
hGH expression in the absence of flanking HGPRT sequences

| CELL | Cell Number | hGH ng/1e6 | Estimated Copy number |
|---|---|---|---|
| STRAIN | (×1e6) | cells/24 h | per haploid genome |
| 136-0.4- 6 | 1.13 | 7208 | 2 |
| 8 | 2.41 | 7066 | 1 |
| 17 | 2.24 | 6248 | 2 |
| 10 | 2.54 | 5816 | 1 |
| HF132-.4-12 | 1.14 | 4454 | 2 |
| 9 | 1.82 | 4173 | 2 |
| 14 | 3.28 | 3374 | 20 |
| 5 | 2.69 | 1971 | 2 |
| 3 | 0.64 | 1969 | 1 |
| 16 | 0.4 | 1578 | 10 |
| 18 | 1.07 | 1568 | 2 |
| 11 | 0.77 | 1568 | 2 |
| 13 | 1.25 | 1378 | 2 |
| 23 | 0.94 | 1371 | 20 |
| 21 | 0.97 | 1350 | 1 |
| 22 | 0.62 | 1042 | 1 |
| 4 | 0.47 | 890 | 1 |
| 13 | 0.61 | 888 | 1 |
| 10 | 0.97 | 874 | 1 |
| 15 | 0.54 | 848 | 1 |
| 20 | 0.89 | 782 | 1 |
| 7 | 1.52 | 780 | 1 |
| 12 | 0.9 | 752 | 2 |
| 15 | 1.42 | 716 | 2 |
| 1 | 0.63 | 616 | 0.5 |
| 17 | 0.69 | 605 | 1 |
| 9 | 0.28 | 603 | 1 |
| 18 | 1.44 | 586 | 1 |
| 5 | 1.06 | 572 | 1 |
| 7 | 1.36 | 555 | 2 |
| 1 | 0.26 | 545 | 1 |
| 19 | 0.85 | 491 | 1 |
| 6 | 0.87 | 466 | 1 |
| 14 | 0.58 | 427 | 1 |
| 2 | 1.02 | 284 | 1 |
| 142-0.4-1 | 1.77 | 188 | 2 |
| 11 | 0.41 | 87 | 2 |
| 16 | 0.52 | 9 | 1 |
| 19 | 0.88 | 3 | 1 |
| Mean | 1.12 | 1659 | 3 |
| Maximum | 3.28 | 7208 | 20 |
| Minimum | 0.26 | 3 | 1 |
| number | 39 | 39 | 39 |

### Example 2. Design of mRNA Constructs for Reverse Gene Therapy

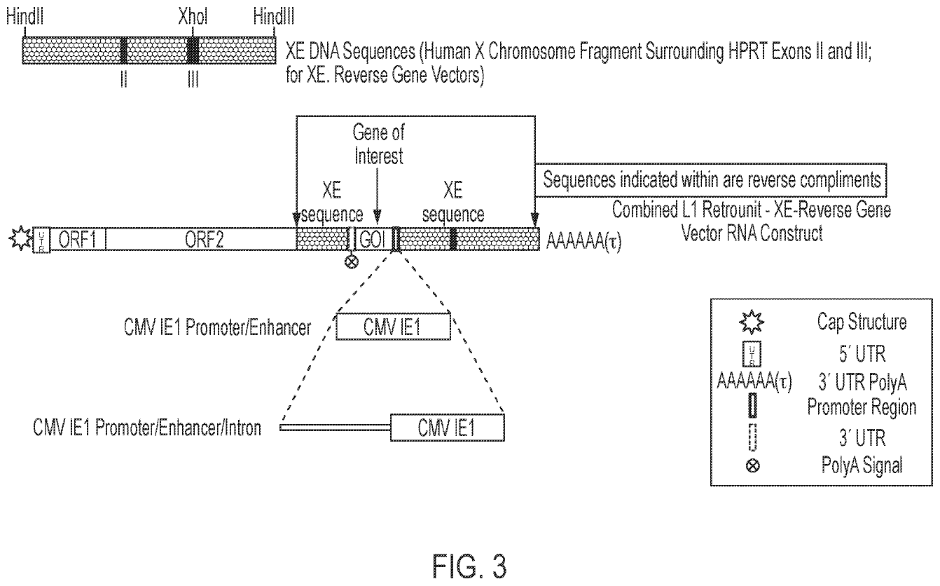

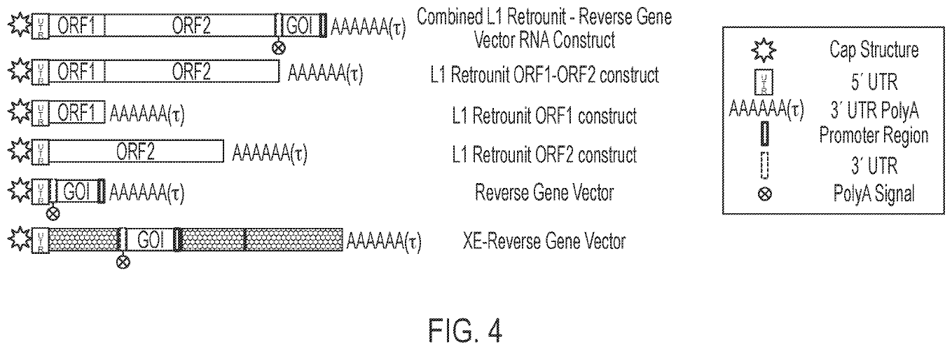

This example illustrates design of exemplary mRNA constructs for reverse gene therapy with a human protein. The reverse gene vector (RGV) is an mRNA construct that includes a codon optimized gene for a protein of interest in the reverse complement orientation. Since the gene of interest is present in reverse, it is “non coding” until it is reverse transcribed. An exemplary single transcript reverse gene vector for RGT also includes XE sequences, a 5′ Cap structure, a 5′ UTR, ORF1, ORF2 endonuclease domain and ORF2 reverse transcriptase domains, a 3′UTR, a 3′ polyA tail as shown in FIG. 3. Multi-transcript compositions of RGT were designed as shown in FIG. 4 such that the L1 retro-elements are present on a transcript different from the reverse gene mRNA. The XE sequences and the gene of interest are present on the transcript as reverse complement sequences.

### Example 3. Exemplary Method for Transgene Delivery

This example provides exemplary methods for effective delivery and expression of RGT mRNA.

Codon optimized RGT mRNA are delivered to cells (e.g., in vivo or ex vivo) using a lipid nanoparticle or exosome. Briefly, aliquots of 50 mg/mL ethanolic solutions of exemplary lipids are mixed and diluted with ethanol to 3 mL final volume. Separately, an aqueous buffered solution of mRNA are prepared. The lipid solution is injected rapidly into the aqueous mRNA solution and mixed to yield a final suspension in 20% ethanol. The resulting nanoparticle suspension is filtered, diafiltrated with 1× PBS (pH 7.4), concentrated and stored at −20° C. Subsequently, encapsulated mRNA is administered to cells ex vivo or to a subject in need of treatment.

### Example 4. Reverse Gene Vector (RGV) RNA Transcripts Drive Stable Retrotransposition (RT) in Human Cells

This example successfully demonstrates that retrotransposition occurs with L1 elements in cis (L1-ORFs on same transcript) or in trans (L1 ORFs and gene of interest on different transcripts) configurations.

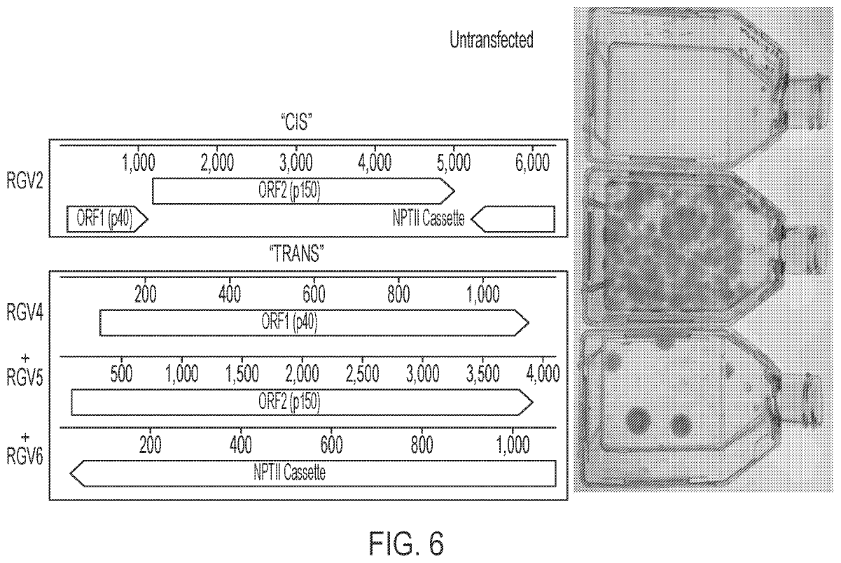

Various reverse gene vectors (RGVs) were prepared. As shown in FIG. 6, RGV2 in cis confirguration comprises L1-ORFs (ORF1 and OFR2) in coding orientation and mRNA construct that includes a codon optimized gene of interest in the reverse complement orientation in the NPTII Cassette. RGV4 and RGV5 each comprises ORF1 and ORF2 in coding orientation, respectively, and RGV6 comprises a gene of interest in the reverse complement orientation.

RGV transcripts were transfected into human cells (typically 5×10⁵ cells) in culture with lipofectamine. For the cis configuration, 2.5 μg of RGV2 was transfected. For trans configuration, total 5 μg of RGV4, RGV5, and RGV6 was co-transfected. The next day following transfection, the cells were trypsinized and plated in medium containing 0.4 mg/ml G418, which allows for selection of cells expressing the NPTII gene. Colonies were visible within 12-14 days and were then either subcultured for further characterization by PCR analysis or fixed and stained with a methylene blue solution. As expected, no colonies were detectable with untransfected cells. FIG. 6 shows that colonies were visible in both cis and trans cells, illustrating that retrotransposition occurs with L1 elements in both cis and trans configurations.

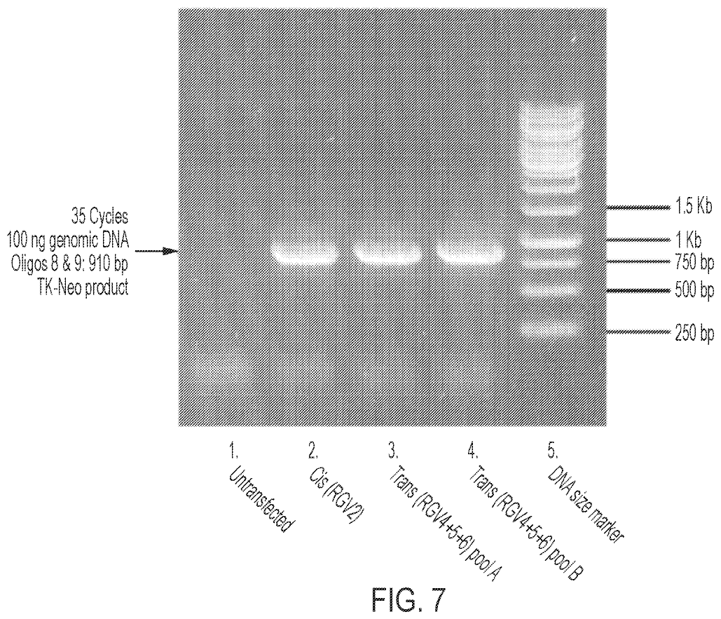

To confirm that the cells contain the gene of interest, PCR analysis was performed. 21 colonies were transferred from the plate of cells transfected with the cis configuration (RGV2), and genomic DNA prepared. 11 colonies were each transferred from the plate of cells transfected with trans configuration (RGV4+5+6), respectively, for pool A and pool B, and genomic DNA prepared. PCR was performed using the standard known method with 35 cycles, and the products were analyzed by gel electrophoresis. As shown in FIG. 7, the expected NPT II gene in human genomic DNA (˜910 bp) following transfection of RGV RNAs in either cis or trans configuration was detected, illustrating that the gene of interest was transposed in the transfected cells.

### Example 5. Reverse Gene Vector (RGV) RNA Transcripts Drive Stable Retrotransposition (RT) in Human Cells without XE Sequences and L1-ORF1

This example successfully demonstrates that retrotranspotision occurs with L1 elements in cis with XE (HPRT) sequences or without XE sequences and L1-ORF1.

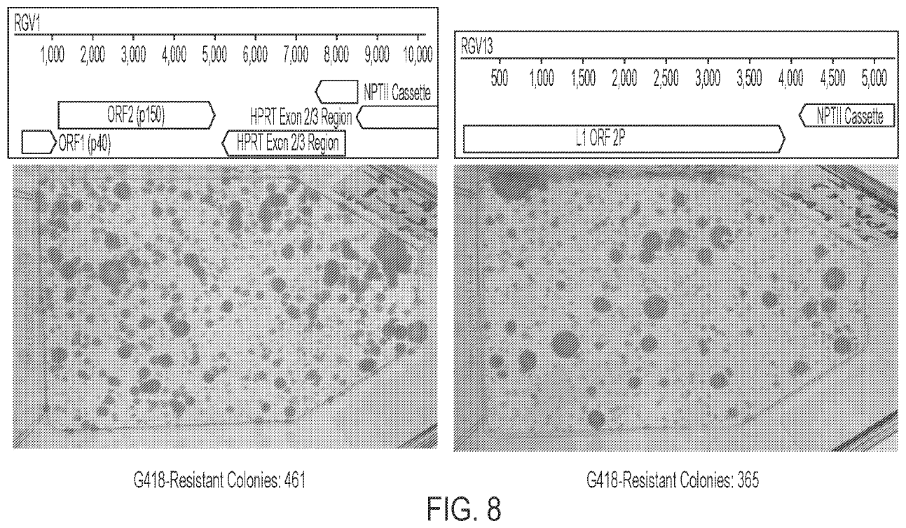

Various reverse gene vectors (RGVs) were prepared. As shown in FIG. 8, RGV1 comprises L1-ORFs (ORF1 and OFR2) in coding orientation and XE sequences (HPRT) and mRNA construct that includes a codon optimized gene of interest in the reverse complement orientation in the NPTII Cassette. RGV13 comprises L1 ORF2 in coding orientation and a gene of interest in the reverse complement orientation, without ORF1 or XE sequences.

Human cells were transfected with either RGV1 or RGV13 (4 μg) with lipofectamine. The next day following transfection, the cells were trypsinized and plated in medium containing 0.4 mg/ml G418, which allows for selection of cells expressing the NPTII gene. Colonies were visible within 12-14 days and were then either subcultured for further characterization by PCR analysis or fixed and stained with a methylene blue solution. As expected, no colonies were detectable with untransfected cells. FIG. 8 shows that colonies were visible in both cells transfected with either RGV1 or RGV13. There was slightly higher number of colonies for cells transfected with RGV1, which contains ORF1 and XE sequences. Overall, this experiment shows that retrotransposition occurs in human cells transfected with RGV that does not comprise XE sequences and ORF1.

### Example 6. Reverse Gene Vector (RGV) RNA Transcripts Drive Protein Expression by Retrotransposition in Human Cells

This example successfully demonstrates that retrotransposed RNA transcripts drive stable protein expression following transfection of RGV RNA.

RGV17 comprising L1 ORF and ORF2 in coding orientation and an mRNA construct encoding human erythropoietin (hEPO) in the reverse complement orientation was prepared. Human cells were transfected with RGV17 (5 μg) with lipofectamine in triplicates (Pool B, B2, and D2). The medium for each pool was sampled for EPO ELISA (R&D Systems) 2 days after plating. EPO ELISA mU/mL values were calculated from the EPO ELISA standard curve. Untransfected control HT1080 cell conditioned media values were subtracted from values from medium of cells transfected with RGV RNAs.

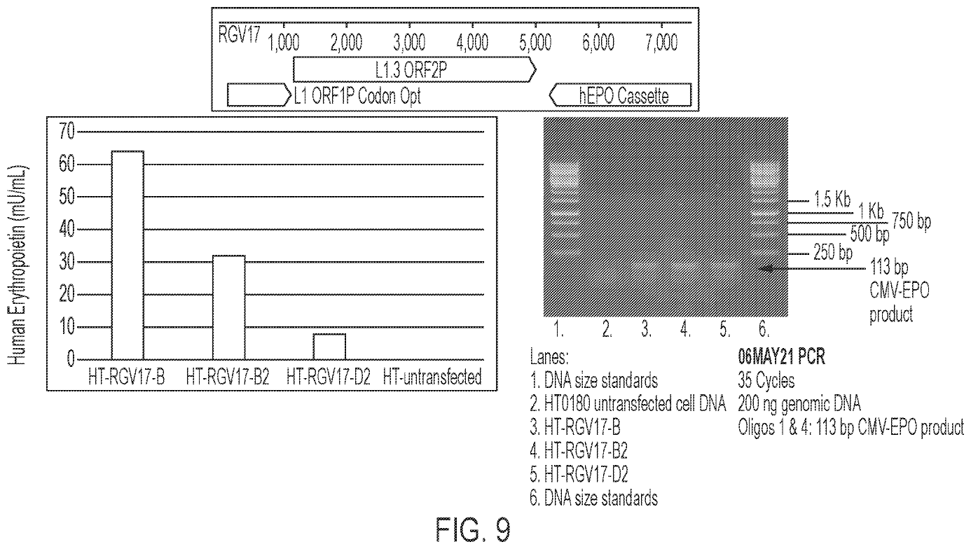

As shown in FIG. 9, hEPO protein expression was detected in all pools, demonstrating that stable hEPO protein expression occurred following transfection of RGV RNA. To confirm that the cells contain the gene of interest, PCR analysis was performed. Colonies were transferred from the plate of cells transfected with RGV17, genomic DNA isolated, and PCR was performed using the standard known method with 35 cycles, and the products were analyzed by gel electrophoresis. As shown in FIG. 9, the expected EPO gene in human genomic DNA (˜113 bp) following transfection of RGV RNAs was detected, confirming that the gene of interest was transposed in the transfected cells.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A composition comprising
a mRNA gene transfer construct comprising a reverse complement sequence encoding a protein of interest; and
a sequence encoding a human L1 retro-element.

2. The composition of claim 1, wherein the gene transfer construct comprises a matrix attachment region (MAR) motif.

3. (canceled)

4. The composition of claim 2, wherein the MAR motif comprises a human X Chromosome fragment surrounding hypoxanthine-guanine phosphoribosyltransferase (HPRT) exons II and III.

5. The composition of claim 2, wherein the gene transfer construct comprises a reverse complement sequence of the MAR motif.

6. The composition of claim 1, wherein the gene transfer construct comprises a eukaryotic origin of replication.

7. The composition of claim 1, wherein the gene transfer construct further comprises a topoisomerase II consensus sequence.

8-9. (canceled)

10. The composition of claim 1, wherein the gene transfer construct comprises a promoter, a enhancer and/or a intron.

11. (canceled)

12. The composition of claim 10, wherein the gene transfer construct comprises a reverse complement sequence of the promoter, enhancer and/or intron.

13. The composition of claim 1, wherein the human L1 retro-element comprises a nuclear localization signal (NLS).

14. (canceled)

15. The composition of claim 1, wherein the human L1 retro-element comprises an ORF2 reverse transcriptase domain.

16. The composition of claim 1, wherein the human L1 retro-element comprises an ORF2 endonuclease domain.

17. The composition of claim 1 wherein the human L1 retro-element comprises an ORF2 reverse transcriptase and does not comprise an ORF2 endonuclease domain.

18. The composition of any claim 1, wherein the human L1 retro-element comprises ORF2 or ORF1.

19-23. (canceled)

24. The composition of claim 1, further comprising a DNA primer for self-priming.

25. The composition of claim 1, further comprising an inverted terminal repeat sequence.

26-28. (canceled)

29. The composition of claim 1, wherein the protein of interest is a therapeutic protein for a single gene deficiency disease, an infectious disease, or cancer.

30. The composition of claim 1, wherein the mRNA gene transfer construct and the sequence encoding a human L1 retro-element are encapsulated in a nanoparticle delivery vehicle.

31. (canceled)

32. The composition of claim 1, wherein the mRNA gene transfer construct and the sequence encoding a human L1 retro-element are encapsulated in an exosome.

33. (canceled)

34. A method of treating a disease comprising administering the composition of claim 1 to a subject in need of treatment.

35. (canceled)

36. A method for gene delivery comprising
administering a mRNA gene transfer construct comprising a reverse complement sequence encoding a protein of interest; and
administering a sequence encoding a human L1 retro-element.

37-43. (canceled)
